(19) United States
(12) Patent Application Publication
(10) Pub. No.: US 2006/0154340 A1
(43) Pub. Date: Jul. 13, 2006

(54) Expression of potato proteinase inhibitor II in microbial hosts

(76) Inventors: Michael Tai-Man Louie, Des Moines, IA (US); Peter Salamone, Modesto, CA (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 11/031,895

(22) Filed: Jan. 7, 2005

(51) Int. Cl. C12P 21/06; C12N 9/99; C12N 1/21; C12N 1/18

(52) U.S. Cl. 435/69.2; 435/184; 435/252.33; 435/254.23

(57) ABSTRACT

Multiple microbial host genetic systems with various molecular constructs enabling subcellular targeting, fusion protein generation and co-expression of ancillary enzymatic activities are used in the expression of potato proteinase inhibitor II. The expression levels achieved exceed previous reports by up to 10 to 1000 fold.

### BACKGROUND OF THE INVENTION

1. Field of the Invention

The invention relates generally to a method for expressing potato proteinase inhibitor II (PI2) in a microbial host and, more specifically, to a method for producing commercial quantities of PI2 using transformed strains of Pichia pastoris and Escherichia coli.

2. Background of the Art

Proteins that inhibit proteolytic enzymes are often found in high concentrations in many seeds and other plant storage organs. Inhibitor proteins are also found in virtually all animal tissues and fluids. These proteins have been the object of considerable research for many years because of their ability to complex with and inhibit proteolytic enzymes from animals and microorganisms. The inhibitors have become valuable tools for the study of proteolysis in medicine and biology. Proteinase inhibitors are of particular interest due to their therapeutic potentials in controlling proteinases involved in a number of disorders such as pancreatitis, shock, and emphysema, and as agents for the regulation of mammalian fertilization. Potato tubers are a rich source of a complex group of proteins and polypeptides that potently inhibit several proteolytic enzymes usually found in animals and microorganisms. In particular, potato inhibitors are known to inhibit human digestive proteinases, and thus have application in the control of obesity and diabetes.

Proteinase inhibitors extracted from potatoes have been distinguished into two groups based on their heat stability. The group of inhibitors that is stable at 80° C. for 10 minutes have been identified as inhibitor I (Melville et al.), carboxypeptidase inhibitor (CPI) (Ryan, C. L., Purification and properties of a carboxypeptidase inhibitor from potatoes. J. Biol. Chem. 249: 5495-5499, 1974), inhibitors IIa and IIb (Bryant, J., Green, T. R., Gurusaddaiah, T., Ryan, C. L. Proteinase inhibitor II from potatoes: Isolation and characterization of its isomer components. Biochemistry 15: 3418-3424, 1976), and inhibitor A5.

Recently, PI2 has been implicated in playing a role in extending satiety in subjects fed a nutritional drink composition containing PI2. U.S. patent application Ser. No. 09/624,922 describes that subjects reported a significant reduction in hunger for up to 3½ hours post meal when fed a meal comprising a nutritional drink composition containing PI2. Likewise, fullness ratings were enhanced, and each study subject lost an average of 2 kg over a 30-day period without experiencing the adverse side effects typically associated with appetite suppressing compounds. Mechanistically, it is thought that as a trypsin and chymotrypsin inhibitor, when consumed by a subject, PI2 stimulates the release of endogenous cholecystokinin, a known hormone peptide effective in reducing the desire to intake food.

The efficiency of oral trypsin/chymotrypsin inhibitor in delaying the rate of gastric emptying in recently diagnosed type II diabetic patients and improving their post-prandial metabolic parameters have been examined (Schwartz, J. G., Guan, D., Green, G. M., Phillips, W. T. Treatment with an oral proteinase inhibitor slows gastric emptying and actually reduces glucose and insulin levels after a liquid meal in type II diabetic patients. Diabetes Care, 17: 255-262, 1994). Serum insulin, plasma glucose, plasma gastric inhibitory polypeptide levels, and the rate of gastric emptying were all significantly decreased over the 2 hour testing period in subjects who received proteinase inhibitor in their oral glucose/protein meal. U.S. Pat. No. 5,187,154 suggests the administration of CCK through an intramuscular injection or an intranasal spray delays gastric emptying and slows delivery of glucose to the blood stream. Alternatively, an oral administration of an agent that enhances endogenous release of CCK could represent an important approach to the treatment of Type 2 diabetes. One of the agents that may have a therapeutic application in patients with recently diagnosed Type 2 diabetes can be the potato proteinase inhibitor II.

Both soybeans and potatoes are sources of proteinase inhibitors (PI's), proteins that have been hypothesized to enhance the release of cholecystokinin (CCK), one of several gut peptides that regulate gastric emptying and satiety in humans (Liddle, R. A. (1995) Am J Physiol 269, G319-27; Beglinger, C. (1994) Ann N Y Acad Sci 713, 219-25; Beglinger, C. (2002) Curr Opin Investig Drugs 3, 587-8). Delayed gastric emptying, in turn, has been shown to result in a decreased rate of glucose absorption, and lower post-prandial glucose levels (Lefebvre, P. J. & Scheen, A. J. (1999) Eur J Clin Invest 29 Suppl 2, 1-6). Proteinase inhibitor II (PI2) is a naturally occurring potent trypsin and chymotrypsin inhibitor present in white potatoes (Melville, J. C. & Ryan, C. A. (1972) J Biol Chem 247, 3445-53; Bryant, J., Green, T. R., Gurusaddaiah, T. & Ryan, C. A. (1976) Biochemistry 15, 3418-24). Previous studies using large doses of highly pure PI2 demonstrated increased CCK release and satiety in humans (Peikin, S. R., Springer, C. J., Dockray, G. J., Blundell, J. E., Hill, A. J., Calam, J. & Ryan, C. A. (1987) Gastroenterology 92, A1570; Hill, A. J., Peikin, S. R., Ryan, C. A. & Blundell, J. E. (1990) Physiol Behav 48, 241-6; Schwartz, J. G., Guan, D., Green, G. M. & Phillips, W. T. (1994) Diabetes Care 17, 255-62). In addition, oral administration of PI2 at high doses in a liquid form has been shown to reduce both post-prandial glucose and insulin levels in humans (Schwartz, et al., supra), supporting the use of PI2 as both a promising hunger management tool and an effective agent to reduce post-prandial glycemia experienced by the body.

Proteinaceous serine protease inhibitors are abundant proteins in the storage organs and seeds of plants belonging to the families of Solanaceae and Leguminosae. In addition, they are an integral part of the defensive mechanisms that protect plants from wounding by pests and bacterial or fungal infections. Potato (Solanum tuberosum) expresses numerous proteinase inhibitors belonging to a wide range of inhibitor families, including the potato proteinase inhibitor II (PI2) family. Members of the PI2 family have been shown to inhibit serine proteases such as trypsin, chymotrypsin, subtilisin, oryzin, and elastase. The cDNA and protein of PI2s isolated from potato tubers have a 2-domain organization; each domain is made up of 54 amino acids. PI2s with 3, 4, or 6 repeats of the 54-amino-acid domain have also been isolated in tomato and tobacco. A high level of protein sequence identity exists among these PI2s, although variations occur among PI2s from different species and among different PI2 isomers of the same species. These variations in sequence, occurring mostly in the reactive site loops of PI2, can give rise to various proteinase inhibitory specificities since it is well known that the specificity of a serine proteinase inhibitor is governed by the reactive site loop

The development of an efficient proprietary commercial process providing an extract from potatoes containing PI2 has increased the availability of this compound. It was hypothesized that administration of PI2 extract as a nutraceutical ingredient in a low dose, encapsulated form, prior to a meal, might reduce post-prandial glucose levels. This could have important implications for the use of PI2 as part of a diet to help maintain healthy blood sugar levels and reduce the propensity for weight gain.

Accordingly, a need exists for a process to produce PI2 in a cost-effective and efficient manner meeting industrial qualitative and quantitative standards.

### SUMMARY OF THE INVENTION

Potato proteinase inhibitor II in commercial quantities has come from the extraction and isolation of the compound from potato tubers. Heterologous expression of PI2 in microbial hosts (Escherichia coli and Pichia pastoris) described in this invention overcomes the limitations of the extraction process. The gene sequence of a PI2 isomer (GenBank Database Accession No. L37519) that has trypsin- and chymotrypsin-inhibiting activities was amplified by PCR from plasmid pE32-(SS-JO). For expression in Escherichia coli, the PCR-amplified PI2 gene was cloned into the commercially available expression plasmid pET32a (from Novagen) as a C-terminal fusion protein of thioredoxin (TrxA), resulting in plasmid pKBEPI-5. Plasmid pKBEPI-5 was then transformed into E. coli BL21 trxB(DE3) for the cytoplasmic production of a TrxA-PI2 fusion protein, after the cells were induced by 1 mM of IPTG. Approximately 32 mg of soluble and active (based on densitometric analysis of a gel) TrxA-PI2 fusion protein was produced in one liter of E. coli cells bearing plasmid pKBEPI-5. Since both TrxA and PI2 are heat stable, a 3-minute heating step at 70° C. precipitated most of the native E. coli proteins from the TrxA-PI2 fusion protein preparation. The internal His-tag between TrxA and PI2 allowed further purification of the TrxA-PI2 fusion protein using a Ni(II)-NTA-Agarose matrix. This yielded a highly pure TrxA-PI2 fusion protein. The TrxA portion of the fusion protein was then removed by an enterokinase treatment, yielding pure PI2.

The advantage of this E. coli expression system is that milligram quantities of PI2 are produced in 1 liter of E. coli cells under a fairly short period of time (1 day) since E. coli is a fast-growing organism. In comparison, a previous attempt to produce PI2 in E. coli yielded only 50 micrograms of PI2 per liter of E. coli cells. An additional advantage is that PI2 can be purified from other E. coli proteins and can be separated from TrxA very efficiently by the method described in this invention.

The essential elements of this invention that result in high-level expression of PI2 in E. coli include: 1) the powerful T7 promoter on plasmid pET32a expresses the trxA-PI2 gene at a high level; 2) the trxA gene of pET32a allows expression of PI2 as a TrxA-PI2 fusion protein. It is believed that the TrxA portion helps PI2 to fold properly and to remain soluble inside E. coli cells. The heat stability of TrxA also allows using simple heating as a purification step to precipitate most of the native E. coli proteins, leaving mostly the TrxA-PI2 fusion protein; 3) the internal His-tag between TrxA and PI2 allows further purification of the fusion protein; 4) the internal enterokinase site allows efficient removal of the TrxA portion from PI2 portion of the fusion protein.

For expression in Pichia pastoris, the PCR-amplified PI2 gene was cloned as a C-terminal fusion with the Sacchromyces cerevisiae mating factor alpha prepro signal peptide (MFα) in the Pichia pastoris expression plasmid pKBPPI-3. A gene coding Zeocin™ (Cayla, Toulouse, France) resistance is present on plasmid pKBPPI-3 that allows for direct selection of P. pastoris transformants containing the expression plasmid. Transcription of the MFα-PI2 gene fusion was under the control of the strong, constitutive glyceraldehyde 3-phosphate dehydrogenase promoter (P_{GAP}) of P. pastoris, located 5′ to the MFα-PI2 gene fusion. A transcriptional terminator of the P. pastoris alcohol oxidase gene (AOX_{TT}) was present 3′ to the MFα-PI2 gene fusion. The expression cassette (PGAP plus MFα-PI2 gene fusion plus AOX_{TT}) was flanked by unique BglII and BamHI sites, and also contained a unique AvrII site within the P_{GAP} region. Site-directed mutagenesis was used to introduce a single base deletion which removed the AvrII site in plasmid pKBPPI-3, resulting in plasmid pKBPPI-3SDM. Then, the expression cassette was released from pKBPPI-3SDM by BglII and BamHI digestion and ligated into BamHI-digested pKBPPI-3. The resulting plasmid, pKBPPI-4, contained 2 tandem copies of the expression cassettes. Plasmid pKBPPI-5 was created by repeating the same procedure 2 more times and contained 4 copies of the expression cassettes.

Cells of P. pastoris KM71H were transformed with AvrII-linearized pKBPPI-5. Transformants were selected on Zeocin-containing media. Zeocin-resistant transformants were then screened for secretion of PI2 in a culture tube experiment. After confirming the production of PI2 from the Zeocin-resistant transformants, one of the transformants, KS4X2, was chosen to test for PI2 production by fed-batch fermentation. Approximately 450 mg of soluble PI2 per liter of fermentation broth was produced. Also, continuous fermentation of strain KS4X2 is feasible since PI2 expression was successfully maintained at 480 to 500 mg per L for 13 days. The N-terminal amino acid sequence of the recombinant PI2 produced by P. pastoris was identical to that of the native PI2, suggesting that the MFα secretion signal was processed properly by P. pastoris cells and was removed from the recombinant PI2 during the protein secretion process.

The advantage of the P_{GAP}-based P. pastoris expression system is that several hundred milligrams of PI2 can be produced in 1 liter of P. pastoris cells. Multiple copies of the PI2-expression cassettes present in one recombinant host significantly contributed to the high expression level. A previous attempt to produce PI2 in P. pastoris by cloning a single copy of the PI2 gene under the control of the methanol-inducible alcohol oxidase promoter (P_{AOX1}) yielded only 11 mg of PI2 per liter. Moreover, expression of PI2 in P. pastoris by the P_{AOX1}-based system has limitations that hinder its development into a large-scale production process. First, expression of PI2 by the P_{AOX1}-based system is a 2-step process. Biomass is generated in the absence of methanol, followed by induction of gene expression in the presence of methanol as the sole carbon source. The time required to achieve peak PI2 concentration is long. In general, the biomass-generating, non-productive stage lasts for 4 to 7 days. Second, accurate regulation of the methanol concentration in the P. pastoris culture during the induction phase is necessary not only to maintain the induction of expression of the PI2 gene but also to prevent accumulation of excess methanol, which is converted to toxic formaldehyde and hydrogen peroxide inside the cells. Third, the methanol-inducible expression system has an extremely high oxygen demand, due to the high cell densities in the fermentor as well as the extra oxygen required for metabolizing methanol. The oxygen demand in methanol-induced P. pastoris fermentations can only be met by sparging the cultures with pure gaseous oxygen, which becomes a major economic and safety concern in large-scale production. Finally, methanol is highly flammable and many production-scale fermentation facilities are not designed to handle the enormous volume of methanol required for large-scale production of recombinant protein by the P_{AOX1}-based system. The constitutive P_{GAP}-based P. pastoris expression system reported in this invention overcomes the above limitations because the hazards and costs associated with the storage and delivery of large volumes of methanol are eliminated. Furthermore, the P_{GAP}-based expression system described in this invention allows expression of PI2 without pure oxygen supplementation. Conventional air sparging was sufficient to sustain the process. The constitutive P_{GAP}-based expression system also allows for continuous production of PI2, saving considerable amount of time and effort in setting up large-scale fermentations run by the fed-batch mode.

The essential elements of this invention that result in high-level expression of PI2 in P. pastoris include: 1) the strong and constitutive PGAP promoter that drives the expression of the MFα-PI2 gene fusion. The P_{GAP} promoter also allows the expression system to be developed into a full-scale production process; 2) the MFα secretion signal efficiently directs secretion of PI2 into the culture medium; 3) multiple copies of the PI2 expression cassette (or PI2 gene) in one P. pastoris strain significantly increases PI2 expression levels when compared to a strain with only 1 copy of the expression cassette.

### BRIEF DESCRIPTION OF THE DRAWINGS

FIG. 1 is an amino acid sequence alignment of PI2_{SS} and PI2_{JO} and wherein the signal peptide sequence that is cleaved off from the mature protein by signal peptidase is underlined.

FIG. 2 is a photograph of SDS-PAGE (A) and Western (B) analyses of BL21trxB(DE3)pLysS cells, containing pKBEPI-1 induced at 37° C.; the arrow indicates the position of PI2_{SS}; MW, molecular weight markers; C, PI2 extracted from potatoes.

FIG. 3 is a photograph of SDS-PAGE (A) and Western (B) analyses of BL21trxB(DE3)pLysS cells, containing pKBEPI-3, induced at 37° C.; the arrow indicates the position of PI2_{JO}; MW, molecular weight markers; C, PI2 extracted from potatoes; WC, E. coli whole cell extracts; sol, soluble fraction of E. coli cell extracts; insol, insoluble fraction of E. coli cell extracts.

FIG. 4 is a photograph of SDS-PAGE analysis of Rosetta-gami(DE3)pLysS cells, containing pKBEPI-3, induced at 37° C.; the arrow indicates the position of PI2_{JO}; MW, molecular weight markers; WC, E. coli whole cell extracts; sol, soluble fraction of E. coli cell extracts.

FIG. 5 is a photograph of SDS-PAGE (A) and Western (B) analyses analysis of BL21trxB(DE3)pLysS cells containing pKBEPI-3 induced at 25° C.; the arrow indicates the position of PI2_{JO}; MW, molecular weight markers; WC, E. coli whole cell extracts; sol, soluble fraction of E. coli cell extracts; insol, insoluble fraction of E. coli cell extracts.

FIG. 6 is a photograph of SDS-PAGE (A) and Western (B) analyses of BL21trxB(DE3)pLysS cells containing pKBEPI-3 induced under different conditions; the arrow indicates the position of PI2_{JO}; WC, E. coli whole cell extracts; sol, soluble fraction of E. coli cell extracts; insol, insoluble fraction of E. coli cell extracts; C, PI2 extracted from potatoes; −ve, whole cell extracts of BL21trxB(DE3)pLysS with no pKBEPI-3.

FIG. 7 is a photograph of SDS-PAGE analysis of TrxA-PI2_{JO} fusion proteins; the arrow indicates the position of the fusion protein; MW, molecular weight markers; sol, soluble fraction of E. coli cell extracts; insol, insoluble fraction of E. coli cell extracts.

FIG. 8 is a photograph of the purification of TrxA-PI2_{JO} fusion protein from BL21trxB(DE3)pKBEPI-5 cell extracts; MW indicates molecular weight markers; lane 1, cell extracts; lane 2, cell extracts after a 3-min incubation at 70° C.; lanes 3, unbound fraction of the Ni²⁺-NTA column; lanes 4 and 5, washes from the Ni²⁺-NTA column; lane 6, TrxA-PI2_{JO} eluted from the Ni-NTA column.

FIG. 9 is a photograph of the enterokinase digestion of Ni²⁺-NTA column purified TrxA-PI2_{JO} fusion protein; MW indicates molecular weight markers; lane 1, an overnight digestion of TrxA-PI2_{JO} by enterokinase at room temperature; lane 2, an overnight incubation of TrxA-PI2_{JO} at room temperature in the absence of any enterokinase.

FIG. 10 is a schematic diagram of the rPI2_{JO} expression plasmid pKBPPI-2. The MFα-PI2_{JO} fusion gene was cloned into the EcoRI and BamHI sites of plasmid pIB2. The labels are: P_{GAP}, the constitutive GAP promoter; AOX_{TT}, AOX1 transcription terminator; Amp^{R}, ampicillin-resistance gene; pUC ori, pUC origin of replication for E. coli; HIS4, histindinol dehydrogenase.

FIG. 11 is a schematic representation of the integration of plasmid pKBPPI-2 into P. pastoris genome at the his4 locus, resulting in transformants that are histidine prototrophs (His⁺).

FIG. 12 is a photograph of SDS-PAGE analysis of the culture supernatants (15 μL) of various His⁺ P. pastoris GS115(his4::pKBPPI-2) clones grown in culture tubes for 3 days. −ve, culture supernatant from the negative control strain GS 115(his4::pIB2).

FIG. 13 is a photograph of a time course SDS-PAGE (A) and Western (B) analyses of the culture supernatants of the constitutive P. pastoris GS115(his4::pKBPPI-2) clone U. Samples were taken from the fermentation #PP12 at the time indicated and 30 μL of the culture supernatants were loaded. nPI2, native PI2 extracted from potatoes. The rPI2_{JO} expression level of the constitutive clone U was compared to the methanol-inducible P. pastoris GS115(P_{AOX1}::pKBPPI-1) clone G25 by SDS-PAGE. Ten μL of culture supernatants of the inducible clone G25 collected at various time during a previous fermentation (#PP03) were loaded onto the SDS-PAGE gel (C).

FIG. 14 is a schematic diagram of (A) Construction of plasmid pKBPPI-4 that carried two tandem copies of the expression cassettes. A unique AvrII site was present only in the first P_{GAP} promoter. Also, plasmid pKBPPI-4 had only 1 BglII site and 1 BamHI site that allow construction of plasmids with multiple copies of expression cassettes by a similar procedure. For example, a plasmid with 4 tandem copies (B) of the expression cassettes, pKBPP-5, was created by sequentially cloning 2 expression cassettes from pKBPPI-3SDM into the unique BamHI site of pKBPPI-4.

FIG. 15 is a photograph of SDS-PAGE analysis of the culture supernatants of various Zeo^{R} transformants of P. pastoris KM71H. Thirty μL of culture supernatants were loaded per lane in lane 2 to 9. Lane 12 was loaded with 15 μL of P. pastoris GS115(his4::pKBPPI-2) culture supernatant. Lane 1, pre-stained broad-range MW standards (BioRad); lanes 2 & 3, transformants of pKBPPI-3, lanes 4 & 5, transformants of pKBPPI-4, lanes 6 & 7, transformants of pKBPPI-5, lanes 8 & 9, transformants of pKBPPI-6; lane 10, pure rPI2_{JO} standard; lane 11, unstained broad-range MW standards (BioRad); lane 12, P. pastoris GS115(his4::pKBPPI-2).

FIG. 16 is a photograph of (A) SDS-PAGE analysis of culture supernatants of fermentations #PP21 and #PP22 sampled at the indicated post-inoculation time (in hours). (B) Western blot of fermentation #PP21 culture supernatants collected at the post-inoculation time indicated (in hours). M, MW standard; rPI2, rPI2_{JO} standard purified from P. pastoris.

FIG. 17 is a graphical representation of rPI2_{JO} concentrations in culture supernatants from fermentations #PP21 (□) and #PP22 (X) as determined by HPLC analysis of resin-purified samples. FIG. 18 is a graphical representation of rPI2JO concentration in culture supernatants of clone KS4X2 cultured in continuous fermentation.

### DETAILED DESCRIPTION OF A PREFERRED EMBODIMENT

I. Expression in Escherichia coli

PI2 has been previously expressed in E. coli at the level of 50 μg·L⁻¹ (Jongsma, M. A., Bakker, P. L., Stiekema, W. J., and Bosch, D. (1995) Mol. Breed. 1, 181-191; Beekwilder, J., Schipper, B., Bakker, P., Bosch, D., and Jongsma, M. (2000) Eur. J. Biochem. 267, 1975-1984). The low-level of expression allows for very limited biochemical analysis and precludes using the expressed protein for biophysical, structural, or clinical studies. To maximize the expression of PI2, the proper expression system must be selected, including the choice of an E. coli host strain with proper genetic background and a vector with optimal features such as origin of replication (regulates copy number, compatibility), promoter, ribosome binding site, fusion partners (regulates targeting, solubility, tagging, purification), multi-cloning site, and selectable marker. Additionally, this proper expression system should be capable of addressing certain expression difficulties associated with the amino acid sequence of PI2, which is set out in Table 1.

TABLE 1
Amino acid percent composition analysis
of the mature PI2_{JO} protein

| | |
|---|---|
| Occurrence % in: | Occurrence % in: |

| Amino | | E. coli | Amino | | E. coli |
|---|---|---|---|---|---|
| Acid | PI2_{JO} | ORFs* | Acid | PI2_{JO} | ORFs* |
| Cys | 12.9 | 1.17 | Ile | 4.0 | 5.98 |
| Gly | 12.1 | 7.34 | Leu | 3.2 | 10.62 |
| Lys | 8.9 | 4.39 | Phe | 3.2 | 3.89 |
| Pro | 8.1 | 4.41 | Arg | 2.4 | 5.52 |
| Ala | 7.3 | 9.45 | Asp | 2.4 | 5.12 |
| Tyr | 7.3 | 2.84 | Met | 1.6 | 2.79 |
| Asn | 6.5 | 3.94 | Gln | 0.8 | 4.41 |
| Glu | 6.5 | 5.72 | His | 0.8 | 2.26 |
| Ser | 6.5 | 5.81 | Val | 0.8 | 7.09 |
| Thr | 4.8 | 5.39 | Trp | 0.0 | 1.52 |
| Total: | 80.6 | 50.46 | | 19.4 | 49.20 |

*Calculated from 4289 E. coli open reading frames (ORFs); data available at the website of the Bioinformatics Center, Institute for Chemical Research at Kyoto University.

Specifically, four amino acids (Cys, Gly, Lys, Pro), account for 42% of the total amino acid content (52/124 AA) in PI2, and these abundant amino acids impart distinctive properties on the polypeptide, including biochemical and structural characteristics that should be taken into account in attempts to over-express PI2. The sheer number of these amino acid residues in the relatively small PI2 polypeptide (124 AA) may also pose difficulties in translation by causing titration of the tRNA pool for these particular amino acids, which may result in early termination of translation and hence poor expression efficiency.

The pET expression system of Novagen possesses many features that are appropriate for over-expressing PI2. The pET system is a powerful system for the expression of recombinant proteins in E. coli. Based on the T7 promoter driven system originally developed by Studier and colleagues (Studier, F. W., Rosenberg, A. H., Dunn, J. J., and Dubendoff, J. W. (1990) Methods Enzymol. 185, 60-89; Studier, F. W., and Moffatt, B. A. (1986) J. Mol. Biol. 189, 113-130; Rosenberg, A. H., Lade, B. N., Chui, D. S., Lin, S. W., Dunn, J. J., and Studier, F. W. (1987) Gene 56, 125-135), the pET system has been used to express thousands of different proteins. The pET system provides numerous possible vector-host combinations that enable tuning of basal expression levels to optimize target gene expression (Rosenberg, et al., 1987). These options are necessary because no single strategy or condition is suitable for every target protein.

Using the pET32a expression vector (Novagen), a high level of expression of PI2 was achieved in the form of a TrxA-PI2 fusion, which can be subsequently cleaved with enterokinase yielding free PI2 and TrxA.

A. Experimental Procedures

Materials. All reagents were of the highest purity available and were purchased from Sigma, Aldrich, or Fisher Scientific. PCR primers were purchased from Integrated DNA Technologies. Taq DNA polymerase, restriction endonucleases, and T4 DNA ligases were purchased from Stratagene, New England Biolabs, and Roche.

Strains, media, and growth conditions. Escherichia coli strain DH5α was used for general cloning purpose. E. coli strainsBL21trxB(DE3), BL21trxB(DE3)pLysS and Rosetta-gami(DE3)pLysS (Novagen) were used as hosts for protein expression. E. coli strains were routinely grown at 37° C. in LB medium (Sambrook, J., Fritsch, E. F., and Maniatis, T. (1989) Molecular cloning: a laboratory manual, 2nd Ed., Cold Spring Harbor laboratory Press, Cold Spring Harbor, N.Y.). Kanamycin, chloramphenicol, and ampicillin were used at 15, 34, and 50 μg·mL⁻¹, respectively, when required. E. coli strains harboring over-expression plasmids were grown at 37° C. in LB medium to a turbidity of 0.5-0.6 at 600 nm before induced by 1 mM isopropyl-β-D-thiogalactopyranoside (IPTG). After induction, cultures were further incubated at 37° C., or at other specific temperatures, with shaking for 3 to 4 h before harvesting the cells for protein expression and solubility analyses.

Cloning the PI2_{SS} gene into plasmidpET25b. Plasmid pE32 (Keil, M., Sanchez-Serrano, J., Schell, J., and Willmitzer, L. (1986) Nucleic Acids Res 14, 5641-5650) and primers pPI2 AseI 5′ S and pPI2 BamHI 3′ AS (Table 2) were used to amplify the PI2_{SS} gene (GenBank accession X04118) by 30 cycles of PCR with a thermal profile of 60 s at 96° C., 60 s at 50° C. and 60 s at 72° C., followed by a 5 min soak at 72° C. and a hold at 4° C. The PCR product was cut by AseI and BamHI and then ligated into the expression plasmid pET25b (Novagen) that was previously digested by NdeI (ends compatible with Asel) and BamHI, producing plasmid pKBEPI-1. SEQ ID NO1 provides the sequence of the PI2_{SS} gene in pKBEPI-1; PI2_{SS} is in bold. After transforming pKBEPI-1 into BL21trxB(DE3)pLysS, native PI2_{SS} would be produced in the cytoplasm of E. coli cells upon induction by IPTG.

TABLE 2

Oligonucleotide primers used for the construction
of various P12 expression vectors

| | |
|---|---|
| Primer | Sequence* |

| | |
|---|---|
| pPI2 AseI 5′ S | 5′-GTCAGTCATTAATGGCGAAGGCTTGCACTTTAG-3′ |
| pPI2 BamHI 3′ AS | 5′-GTCAGTCGGATCCTACATTGCAGGGTACATATTTG-3′ |
| pPI2 NcoI 5′ S | 5′-GTCAGTCCCATGGCGAAGGCTTGCACTTTAG-3′ |
| oSPSDM1 | 5′-TTGTGAAGGAGAGTCTGACCCAAAAAAA |
| | CCAAAAGCATGCCCCCGGAATTGCGATC-3′ |
| oSPSDM2 | 5′-GATCGCAATTCCGGGGGCATGCTTTTGGTT |
| | TTTTTGGGTCAGACTCTCCTTCACAA-3′ |
| pPI2 seq S1 | 5′-TGTGGTAATCTTGGGTTTGG-3′ |
| pPI2 seq AS1 | 5′-GGGCTCATCACTCTCTCC-3′ |
| TrxA BamHI 5′ S | 5′-GTCACTCGGATCCAAGAAGGAGATATACATATGAGC-3′ |
| TrxA SalI 3′ AS | 5′-CAGTCAGGTCGACTCAGCCAGAACCAGAACCGGCCAG-3′ |

*The bold face indicates restriction sites.
Underlined sequences were 5′ overhangs designed for facilitating restriction digestion at the ends of the PCR products.

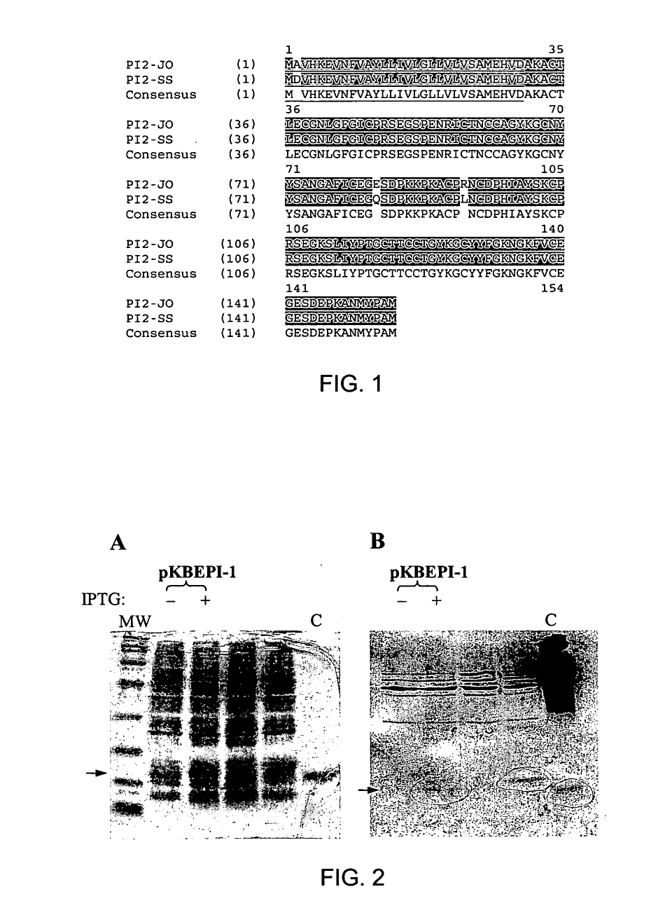

Site-directed mutagenesis of the PI2_{SS} gene to the PI2_{JO} gene and cloning of the PI2_{JO} gene. The PI2_{SS} gene was mutated to the PI2_{JO} gene sequence (GenBank accession L37519) by using the Quikchange site-directed mutagenesis kit (Stratagene) according to the manufacturer's instruction. Briefly, plasmid pE32 and primers oSPSDM1 and oSPSDM2 (Table 2) were used to introduce 2 mutations, changing the mature PI2_{SS} protein sequence from Gln⁵² to Glu⁵² and from Leu⁶³ to Arg⁶³. This doubly mutated PI2_{SS} sequence, identified as plasmid pE32-(SS-JO), was identical to that of PI2_{JO} (FIG. 1), and was confirmed by DNA sequencing with primers pPI2 seq S1 and pPI2 seq AS1 (Table 2). The PI2_{JO} gene was then PCR-amplified with primers pPI2 AseI 5′ S plus pPI2 BamHI 3′ AS (Table 2) and cloned into NdeItBamHI-cut pET25b, resulting in plasmid pKBEPI-3. SEQ ID NO₂ provides the sequence of the PI2_{JO} gene sequence (bold) in pKBEPI-3. Plasmids pKBEPI-3 was subsequently transformed into E. coli BL21trxB(DE3)pLysS and E. coli Rosetta-gami(DE3)pLysS for protein expression upon induction by IPTG.

Construction of trxA-PI2_{JO} fusion gene. Plasmid pKBEPI-3 and primers pPI2 NcoI 5′ S plus pPI2 BamHI 3′ AS (Table 2) were used to amplify the PI2_{JO} gene by PCR. PCR-amplified PI2_{JO} gene that was cut by NcoI and BamHI was subcloned into NcoI/BamHI-digested plasmids pET32a (trxA-fusion) (Novagen), creating plasmid pKBEPI-5. SEQ ID NO₃ provides the sequence of the trxA-PI2_{JO} gene sequence in pKBEPI-5; the trxA tag is underlined and PI2_{JO} is bold. Plasmid pKBEPI-5 was transformed into BL21trxB(DE3) and Rosetta-gami(DE3)pLysS for protein expression.

Cell extract preparation. After induction, E. coli cells were harvested by centrifugation at 10,000×g for 10 min and suspended in 5 to 10 mL of 50 mM potassium phosphate (KPi) buffer (pH 7.0) containing 0.1 mg·mL⁻¹ lysozyme, and 5 mM EDTA. The cells were incubated at room temperature for 30 min and followed by a 15-min freeze-thaw cycle at −80° C. and 37° C. The cells were further disrupted by two 30-second pulse sonications (Sonic Dismembrator 60, Fisher Scientific) at 4° C. The lysate was then centrifuged at 30,000×g for 10 min, and the supernatant was saved as cell extract.

Purification of TrxA-PI2_{JO} protein. TrxA-PI2_{JO} protein was purified from 10 mL cell extract prepared from a 50-mL, IPTG-induced E. coli BL21trxB(DE3)pKBEPI-5 culture. The cell extract was heated at 70° C. for 3 min to precipitate heat labile non-TrxA-PI2_{JO} proteins. After dialyzing the heat-treated cell extracts (MWCO 3,500) against 1 L of 25 mM KPi buffer (pH 7.0) with 250 mM NaCl for the removal of EDTA, TrxA-PI2_{JO} was purified using a Ni²⁺-NTA-Agarose matrix (Qiagen) according to the manufacturer's instruction. Briefly, 2 mL of the dialyzed cell extract was mixed with 1 mL of Ni²⁺-NTA-Agarose matrix and 20 mM imidazole for 1.5 h at 4° C. The mixture was packed into a small column, and the column was washed twice with 5 mL 25 mM KPi buffer (pH 7.0) with 250 mM NaCl and 20 mM imidazole. TrxA-PI2_{JO} was then eluted from the column with 5 mL of the same buffer containing 250 mM imidazole.

Analytical methods. Protein concentrations were determined with a protein dye reagent (Bradford, M. M. (1976) Anal. Biochem. 72, 248-254) (BioRad) with a bovine serum albumin solution (Pierce) as a standard. SDS-PAGE was done by the method of Laemmli (Laemmli, U. K. (1970) Nature 227, 680-685), and gels were stained for proteins with GelCode Blue (Pierce). For Western analysis, SDS-PAGE gels were electroblotted onto a nitrocellulose membrane (BioRad) using Towbin buffer (Towbin, J., Staehelin, T., and Gordon, J. (1979) Proc. Natl. Acad. Sci. USA 76, 4350-4354). Polyclonal rabbit anti-tomato-PI2 antibody (a gift from Dr. C. Ryan, Washington State University) was used to hybridize to the blot for 16 h in Tris-buffered saline (Sambrook, et al. 1989) with 5% (w/v) dry milk. The blot was then developed by using alkaline phosphatase-conjugated goat anti-rabbit antibodies (BioRad) in 10 mL of a 100 mM Tris-HCl buffer (pH 9.5) containing 100 mM NaCl, 5 mM MgCl₂, 0.33 mg·mL⁻¹ nitro blue tetrazolium, and 0.165 mg·mL⁻¹ 5 bromo-4-chloro-3-indolylphosphate. N-terminal amino acid sequencing was performed by electroblotting SDS-PAGE-resolved PI2 proteins onto a polyvinylidene difluroide membrane (BioRad) using a 10 mM CAPS buffer (pH 11) with 10% (v/v) methanol. The protein blot was stained by GelCode Blue. The PI2 band was then excised from the blot for N-terminal sequencing at the Nucleic Acid-Protein Service Unit at the University of British Columbia. Trypsin inhibition activity was measured by combining a known amount of trypsin with a substrate, Nα-p-Tosyl-L-arginine methyl ester (TAME), which is hydrolyzed to p-Toluenesulfyl-L-arginine (TSA). The absorbance at 247 nm was kinetically monitored to measure the amount of TSA released by trypsin. Addition of different dilutions of PI2 resulted in different rates of trypsin activity due to inhibitor binding to trypsin. These rates were plotted to result in an inhibition curve. One unit of trypsin inhibitor activity is defined as the amount causing 50% inhibition of trypsin.

B. Results and Discussion

Cloning and expression of the PI2_{SS} gene in strain BL21trxB(DE3)pLysS. Several PI2 isomers that differ slightly in amino acid sequences exist naturally in potatoes. The gene of one of the isomers, PI2_{SS}, was cloned into expression plasmid pET25b. Plasmid pKBEPI-1 is designed to produce native PI2_{SS} protein in the cytoplasm of BL21 trxB(DE3)pLysS. The trxB mutation in the expression host has been shown to facilitate cytoplasmic disulfide bond formation (Derman, A. I., Prinz, W. A., Belin, D., and Beckwith, J. (1993) Science 262, 1744-1747.), which is important for the correct folding of PI2 molecules. After induction by IPTG, a protein with apparent molecular weight similar to native PI2_{SS} (FIG. 2A) was produced. Western analyses confirmed that the induced protein was PI2_{SS} (FIG. 2B). The apparent molecular weight of the recombinant native PI2_{SS} is noticeably larger than that of native PI2 extracted from potatoes (FIGS. 2A&B). The reason for this apparent discrepancy is unclear but it is reproducible and thus is not an SDS-PAGE artifact. In addition, this discrepancy is likely not due to post-translational glycosylation of the recombinant PI2_{SS} since E. coli cells usually do not glycosylate cytoplasmic proteins. The N-terminal amino acid sequence of PI2 extracted from potatoes was determined to be AKACTLECGN, which is identical to the reported N-terminus of mature PI2_{SS} (FIG. 1).

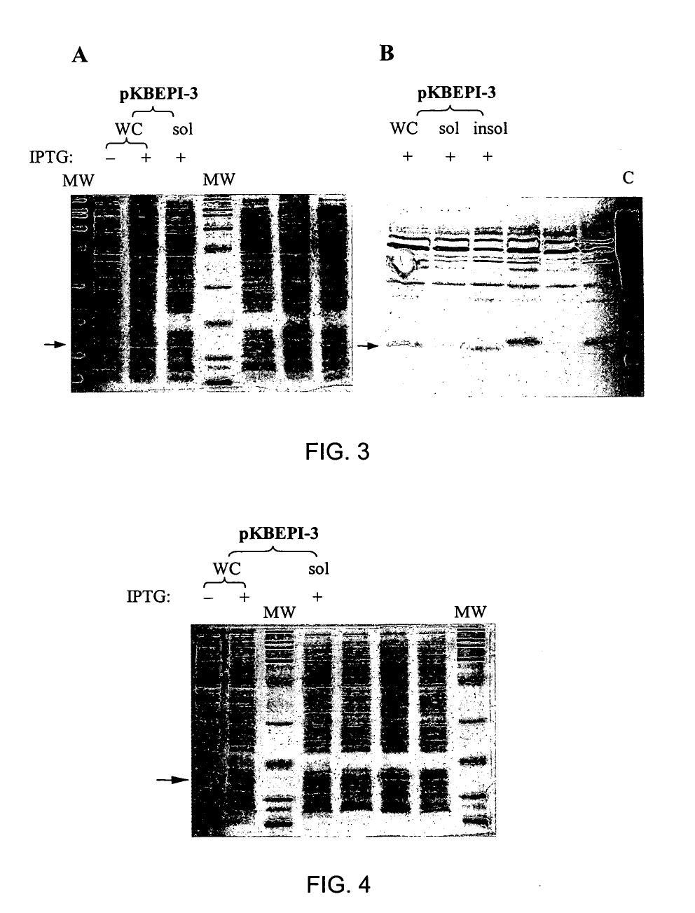

Mutagenesis of the PI2_{SS} gene to PI2_{JO} gene and its expression. The PI2_{SS} gene sequence was altered by site-directed mutagenesis to the PI2_{JO} gene sequence, another natural PI2 isomer. Beekwilder et al. (Beekwilder, J., Schipper, B., Bakker, P., Bosch, D., and Jongsma, M. (2000) Eur. J. Biochem. 267, 1975-1984) previously reported the trypsin inhibitory activity of PI2_{JO} is approximately 1.4-fold of the chymotrypsin inhibitory activity (based on Kᵢ values). The co-existence of similar trypsin and chymotrypsin inhibitory activities in the same PI2 molecule is believed to be critical for the satiety effect of PI₂. The PI2_{JO} gene was then cloned into pET25b forming plasmid pKBEPI-3. PI2_{JO} was successfully produced in E. coli as shown by SDS-PAGE and Western analyses (FIG. 3).

One factor that may limit the expression level of PI2_{JO} in E. coli is rare codon usage in PI2_{JO} gene sequence. An examination of the PI2_{JO} gene sequence did not identify any particular rare codon that would limit PI2_{JO} expression in E. coli (Table 3). However, PI2_{JO} is made up of an extremely high number of several amino acids that include Cys, Lys, Tyr, and Pro (Table 1). Examination of 4,289 E. coli ORFs reveals the normal usage frequencies of these 4 amino acids were ˜2 to 11-fold greater in the coding sequence of PI2 (Table 1). Under typical growth conditions, the resident tRNA population available for protein synthesis would more or less resemble the amino acid usage frequency of different amino acids. Thus, high-level expression of PI2_{JO} in E. coli could be limited at translational level due to the shortage of tRNAs for the abundant amino acids in PI2_{JO}. In order to alleviate this potential problem, expression of PI2_{JO} from plasmid pKBEPI-3 was examined in strain Rosetta-gami(DE3)pLysS, which carries extra copies of tRNA genes for Pro and Tyr on the multi-copy plasmid pLysS. Furthermore, this strain is a trxB/gor double mutant that would greatly enhance cytoplasmic disulfide bond formation (Prinz, W. A., Aslund, F., Holmgren, A., and Beckwith, J. (1997) J. Biol. Chem. 272, 15661-15667.; Derman, et al.). SDS-PAGE analysis showed that expression of PI2_{JO} in Rosetta-gami(DE3)pLysS was not significantly enhanced (FIG. 4) when compared to those in BL21trxB(DE3)pLysS (FIG. 3). The enhancement might have been more noticeable if extra tRNA genes for Cys (the most abundant amino acids in PI2_{JO}) had been cloned into pLysS. Most of the PI2_{JO} was still synthesized as insoluble inclusion bodies so several approaches were carried out to attempt to improve the solubility of PI2_{JO} when expressed in E. coli.

TABLE 3
Analysis of rare E. coli codons in the PI2_{JO} gene sequence.

| Rare | Amino | Frequency of the rare codon | Total number of the |
|---|---|---|---|
| E. coli | acid | present in PI2_{JO} gene | particular amino acid |
| codon | encoded | sequence | in PI2_{JO} |
| AGG | Arg | 0 | 3 |
| AGA | Arg | 0 | 3 |
| CGA | Arg | 0 | 3 |
| CGG | Arg | 0 | 3 |
| AUA | Ile | 2 | 5 |
| CUA | Leu | 2 | 4 |
| CCC | Pro | 4 | 10 |
| UCG | Ser | 1 | 8 |

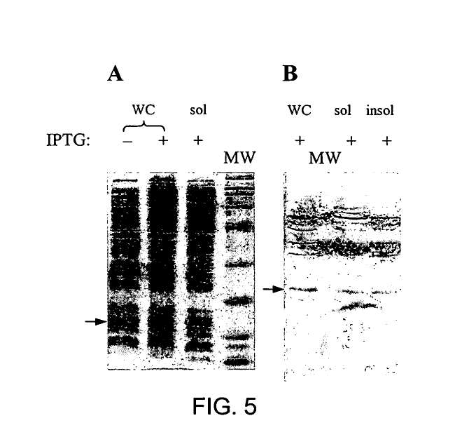

Modification of culture conditions to improve PI2_{JO} solubility in E. coli. To produce soluble, heterologous protein in E. coli, various approaches have been developed, including lowering the culture temperature (Bishai, W. R., Rappuoli, R., and Murphy, J. R. (1987) J. Bacteriol. 169, 5140-5151; Steczko, J., Donoho, G. A., Dixon, J. E., Sugimoto, T., and Axelrod, B. (1991) Protein Expr. Puri. 2, 221-227), applying different types of stress to the cultures (Steczko, et al.; Blackwell, J. R., and Horgan, R. (1991) FEBS Lett. 295, 10-12), and reducing the redox potential of the culture. The effect of lowering the cultivation temperature after induction to 25° C. was examined (FIG. 5). The production level of PI2_{JO} at 25° C. decreased significantly compared to that at 37° C. (FIG. 5 vs. FIG. 3). But the distribution of PI2_{JO} in different fractions was shifted according to Western analysis. The ratio of PI2_{JO} in the soluble fraction to that in the insoluble fraction shifted from 1:3 at 37° C. (FIG. 3B) to approximately 1:1 at 25° C. (FIG. 5B).

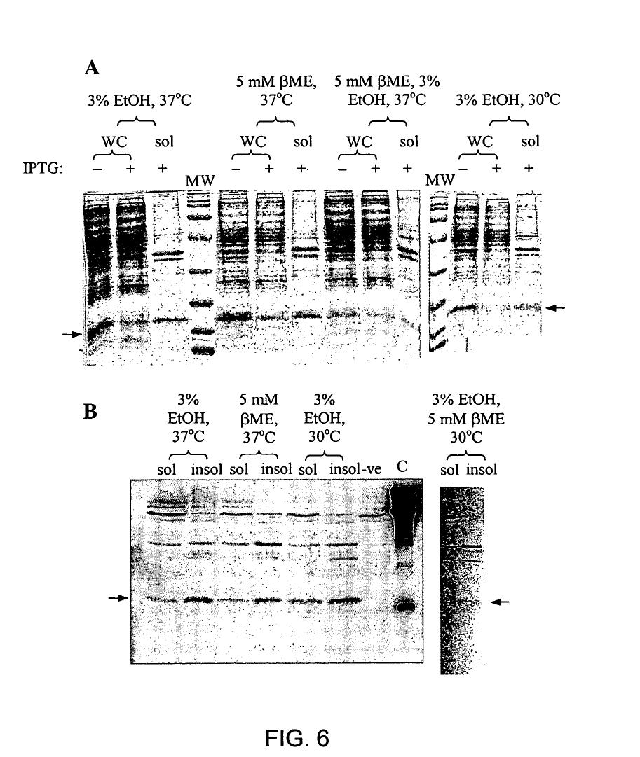

Steczko et al. have shown that addition of ethanol to E. coli cultures can reduce the production of over-expressed proteins as inclusion bodies. Therefore, 3% ethanol (v/v) was added to a PI2_{JO}-producing BL21trxB(DE3)pLysS culture at 37° C. The level of PI2_{JO} production was higher than that of the same culture grown at 25° C. without ethanol addition, but the distribution of PI2_{JO} in the soluble and insoluble fractions remained close to 1:3 (FIG. 6). The combinatorial effect of cultivation temperature and ethanol was investigated. About 40% of the PI2_{JO} remained as soluble proteins in a culture grown at 30° C. with 3% ethanol (FIG. 6) and the total level of expression was not significantly lower than that at 37° C. with 3% ethanol (FIG. 6). Meanwhile, addition of 5 mM P-mercaptoethanol to a BL21trxB(DE3)pLysS/pKBEPI-3 culture at 37° C. did not appear to have any effect on the solubility of PI2_{JO} because the distribution of PI2_{JO} in the soluble and insoluble fractions remained close to 1:3 (FIG. 6). When 5 mM β-mercaptoethanol plus 3% ethanol were added to the culture, the cells produced almost no soluble PI2_{JO} (FIG. 6).

In summary, lowering the cultivation temperature to 30° C. plus adding 3% ethanol to the induced culture significantly improved the solubility of PI2_{JO} produced in E. coli BL21trxB(DE3)pLysS. However, the production level is not high enough to warrant using this strategy for producing PI2_{JO} for clinical research or commercial production. Therefore, a different approach was sought to improve both the productivity and solubility of PI2_{JO}.

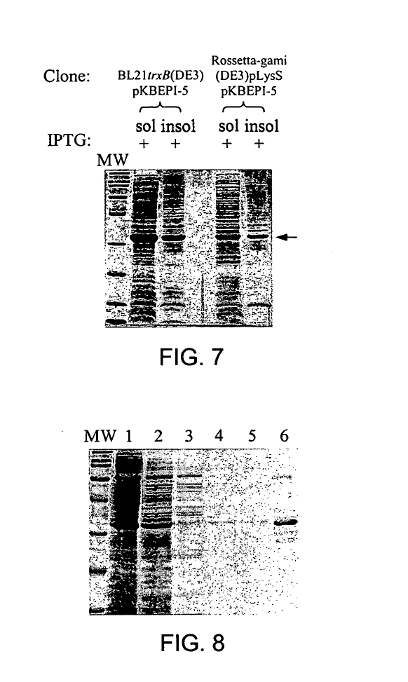

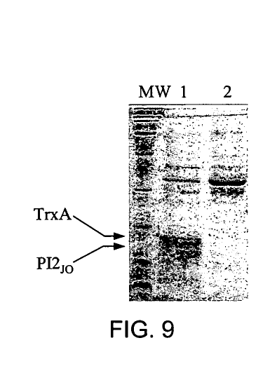

High-level Production of PI2_{JO} as a soluble TrxA-PI2_{JO} fusion protein. Thioredoxin (TrxA) has been stably expressed at very high level and is extremely soluble in the E. coli cytoplasm (Lunn, C. A., Kathju, S., Wallace, B. J., Kushner, S. R., and Pigiet, V. (1984) J. Biol. Chem. 259, 10469-10474). Additionally, a number of eukaryotic proteins, when expressed as C-terminal TrxA-fusion proteins, also stayed remarkably soluble in the E. coli cytoplasm (Lauber, T., Marx, U. C., Schulz, A., Kreutzmann, P., and Rosch, P. (2001) Protein Expr. Puri. 22, 108-112; Jiang, S. T., Tzeng, S. S., Wu, W. T., and Chen, G. H. (2002) J. Agri. Food Chem. 50, 3731-3737; LaVallie, E. R., DiBlasio, E. A., Kovacic, S., Grant, K. L., Schendel, P. F., and McCoy, J. M. (1993) Bio/Technology 11, 187-193). Consequently, an expression plasmid, pKBEPI-5, was constructed in which PI2_{JO} was fused to the C-termini of TrxA. Plasmid pKBEPI-5 was transformed into strains BL21trxB(DE3) and Rosetta-gami(DE3)pLysS for the cytoplasmic production of TrxA-PI2_{JO} fusion. Since pLysS containing strains produce cytoplasmic lysozyme, an inhibitor of T7 RNA polymerase, as a stringent transcriptional control factor for toxic gene expression, strain BL21trxB(DE3)pLysS was not used because expression of PI2_{JO} did not appear to be toxic to E. coli cells. SDS-PAGE analyses showed that a large amount of TrxA-PI2_{JO} stayed in the soluble fraction of strains BL21 trxB(DE3) and Rosetta-gami(DE3)pLysS (FIG. 7). A 10-fold concentrated cell extract from a 50 mL BL21trxB(DE3)pKBEPI-5 culture had 1390 Trypsin-Inhibition-Unit/mL. A densitometric analysis of the soluble TrxA-PI2_{JO} band (FIG. 7) showed that the TrxA-PI2_{JO} band was on average 3.5 times darker than the bands (1 μg each) in the molecular weight standard. Thus, the production of TrxA-PI2_{JO} by BL21trxB(DE3)pKBEPI-5 was estimated to be 32 mg/L. Since both TrxA (Hiraoki, T., Brown, S. B., Stevenson, K. J., and Vogel, H. J. (1988) Biochem. 27, 5000-5008) and PI2_{JO} (results not presented) were heat stable, purity of the TrxA-PI2_{JO} protein could be improved by a 3-min heating at 70° C. (FIG. 8, lane 2). Also, the internal 6×His-tag between TrxA and PI2_{JO} allowed further purification of TrxA-PI2_{JO} proteins using a Ni²⁺-NTA-Agarose matrix (FIG. 8, lane 6). About 15 mg of TrxA-PI2_{JO} could be purified from a liter of BL21trxB(DE3)pKBEPI-5. PI2_{JO} can be separated from the TrxA portion by an enterokinase treatment (FIG. 9).

C. Summary

The results of different expression constructs reported in this study are summarized in Table 4. Among all of these constructs, an E. coli host expressing a TrxA-PI2_{JO} fusion gene produced a large amount of soluble and active TrxA-PI2_{JO} fusion protein. The expression level of TrxA-PI2_{JO} fusion protein was almost 3 orders of magnitude higher than that previously reported by Jongsma et al and Beekwilder-et al. This fusion protein is easily purified and the TrxA fusion tag can be removed by an enterokinase treatment, producing a pure PI2 isomer. The fast growth rate of E. coli and the quantitative recovery of a pure PI2 isomer make this expression system an attractive candidate for production of pure PI2 isomer.

TABLE 4
Summary of the different E. coli hosts expressing the PI2 gene.

| | | | Special | | | | |
|---|---|---|---|---|---|---|---|
| | | | feature | | | | |
| | | PI2 | in the | Question | Expression | % | |
| Vector | Host | gene | gene | asked | level | soluble | Comments |
| pKBEPI-1 | BL21trxB(DE3)p | PI2_{SS} | — | Production | + | below | — |
| | LysS | | | in E. coli | | 5% | |
| | | | | cytoplasm? | | | |
| pKBEPI-3 | BL21trxB(DE3)p | PI2_{JO} | — | Production | + | ca. | — |
| | LysS | | | in E. coli | | 40% at | |
| | | | | cytoplasm? | | 30° C. | |
| | | | | | | with | |
| | | | | | | 3% | |
| | | | | | | ethanol | |
| pKBEPI-3 | Rosetta- | PI2_{JO} | — | Extra copies | + | below | — |
| | gami(DE3)pLysS | | | of rare | | 5% | |
| | | | | codon | | | |
| | | | | improve | | | |
| | | | | cytoplasmic | | | |
| | | | | expression? | | | |
| pKBEPI-5 | BL21trxB(DE3) | PI2_{JO} | TrxA- | Increase | +++++ | ca. | — |
| | | | fusion | cytoplasmic | | 50% | |
| | | | | expression | | soluble | |
| | | | | and | | | |
| | | | | solubility by | | | |
| | | | | TrxA- | | | |
| | | | | fusion? | | | |
| pKBEPI-5 | Rosetta- | PI2_{JO} | TrxA- | Increase | +++ | ca. | The T7 |
| | gami(DE3)pLysS | | fusion | cytoplasmic | | 50% | lysozyme |
| | | | | expression | | soluble | produced |
| | | | | and | | | by pLysS |
| | | | | solubility by | | | suppressed |
| | | | | TrxA- | | | production. |
| | | | | fusion? | | | |

II. Constitutive Expression in Pichia pastoris

This experiment describes the development of a P. pastoris strain for the production of PI2 using the glyceraldehyde-3-phosphate dehydrogenase promoter (PGAP) system.

A. Experimental Procedures

Materials. All reagents were of the highest purity available and were purchased from Sigma, Aldrich, and Fisher Scientific unless otherwise noted. PCR primers were purchased from Integrated DNA Technologies. Taq DNA polymerase, restriction endonucleases, and T4 DNA ligases were purchased from Invitrogen, New England Biolabs, and Roche, respectively.

Host strains and media. Escherichia coli strain DH5α was used for general cloning purpose. E. coli strains were routinely grown at 37° C. in LB media (Sambrook, et al. 1989) Ampicillin and Zeocin were used at 50 μg·mL⁻¹ and 25 μg·mL⁻¹, respectively, when required. Pichia pastoris strain GS 115 (His⁻, where His⁻ represents an auxotrophic phenotype that requires histidine supplementation) (Invitrogen) was used for PI2 expression. P. pastoris GS115 was normally cultured in YPD medium (1% (w/v) yeast extract, 2% (w/v) peptone, 2% (w/v) dextrose). BMGS plates with no histidine (100 mM potassium phosphate (pH 6.0), 1.34% (w/v) yeast nitrogen base, 4×10⁻⁵% (w/v) biotin, 1 M sorbitol, 2% (w/v) agar) were used for selection of P. pastoris GS115 cells transformed with the PI2 expression plasmid.

Cloning the PI2_{JO} gene into plasmidpPICZαA. Plasmid pE32-(SS-JO), and primers pPI2 XhoI 5′S (5′-GTCAGTCCTCGAGAAAAGAGCGAAGGCTTGCACTTTAG-3′) plus pPI2 XbaI 3′ AS (5′-CAGTCATCTAGATCACTACATTGCAGGGTACATATTTG-3′) were used for amplifying the PI2_{JO} gene (GenBank accession L37519) by 30 cycles of PCR with a thermal profile of 30 s at 95° C., 30 s at 58° C. and 60 s at 72° C., followed by a 10-min soak at 72° C. and a hold at 4° C. The PCR product was cut by XhoI and XbaI and then ligated into the expression plasmid pPICZαA (Invitrogen) that was previously digested by XhoI and XbaI, producing plasmid pKBPPI-1. After transforming pKBPPI-1 into E. coli DH5α cells for propagation of the plasmid, the PI2_{JO} sequence in pKBPPI-1 was confirmed by DNA sequencing, using sequencing primer a-Factor seq (5′-TACTATTGCCAGCATTGCTGC-3′).

Cloning the MFα-PI2_{JO} fusion gene into plasmidpIB2. Primers MFα-PI2-F2 5′ S (5′-GTCAGTCGAATTCCGAAACGATGAGATTTCCTTCA-3′) plus pPI2 BamHI 3′ AS (5′-GTCAGTCGGATCCTACATTGCAGGGTACATATTTG-3′) were used for amplifying the MFαPI2_{JO} fusion gene (where MFα is the secretion signal peptide of Saccharomyces cerevisiae mating factor alpha 1) from plasmid pKBPPI-1 by 30 cycles of PCR with a thermal profile of 30 s at 95° C., 30 s at 58° C. and 60 s at 72° C., followed by a 10 min soak at 72° C. and a hold at 4° C. The PCR product was cut by EcoRI and BamHI and then ligated into the expression plasmid pIB2 (Sears, I. B., O'Connor, J., Rossanese, O. W., and Glick, B. S. (1998) Yeast 14, 783-790) that was previously digested by EcoRI and BamHI, producing plasmid pKBPPI-2 (FIG. 11). SEQ ID NO4 provides the sequence of the MFa-PI2 fusion gene sequence in pKBPPI-2. Plasmid pKBPPI-2 was transformed into E. coli DH5α cells for propagation of the plasmid.

P. pastoris transformation and culture tube expression experiment. P. pastoris strain GS115 was transformed with ca. 250 ng of SalI-linearized pKBPPI-2 by electroporation as reported by Sears et al. In a separate experiment, GS115 cells were transformed with 250 ng of SalI-linearized pIB2. Transformants from both experiments were selected on BMGS plates with no histidine. After three days of incubation at 30° C., possible His⁺ transformants were streaked for purity on BMG plates (same as BMGS except the medium did not contain sorbitol). His⁺ transformants from the pKBPPI-2 transformation were then screened for possession of the MFα-PI2_{JO} fusion gene by colony PCR (Linder, S., Schliwa, M., and Kube-Granderath, E. (1996) BioTechniques 20, 980-982) using primers a-Factor seq (5′-TACTATTGCCAGCATTGCTGC-3′) and 3′ AOX1 seq (5′-GCAAATGGCATTCTGACATCC-3′). Positive transformants were then tested for recombinant PI2_{JO} (rPI2_{JO}) secretion. A His⁺ transformant from the pIB2 vector only transformation was chosen as a non-rPI2-producing negative control. Twenty μL of cells that had grown overnight in YPD medium at 30° C. were used to inoculate 5 mL fresh YPD media. The cultures were incubated at 30° C. with shaking at 270 rpm for 3 days. Every 24 h, 100 μL of the cultures were sampled to analyze for rPI2_{JO} production by SDS-PAGE analysis.

Constitutive expression of rPI2_{JO} by fermentation. One of the positive transformants that produced rPI2_{JO} in the culture tube experiment, designated as clone U, was chosen to test for rPI2_{JO} production under fermentative conditions. A single colony isolate of clone U was used to inoculate 100 mL YPD and was incubated for 23 h at 30° C., 200 rpm. Three mL of this culture was used to inoculate 300 mL YPD. The seed culture was grown at 30° C., 200 rpm for 22-24 h. This seed culture was used to inoculate a 14-L BioFlo 3000 vessel (New Brunswick Scientific Co.) equipped with two Rushton impellers and four baffles, containing 8 L Basal Salt Medium, 40 g·L⁻¹ Cerelose® (industrial scale dextrose, Corn Products International), 0.8 mg·L⁻¹ d-biotin, and 1×PTM1 trace element solution (Stratton, J, Chiruvolu, V, and Meagher, M. (1998) in Pichia protocols (Higgins, D. R., and Cregg, J. M., eds) Vol. 103, pp. 107-120, Humana Press Inc., Totowa, N.J.). The production culture temperature was maintained between 23° C.-30° C. for fermentations #PPI2 and #PP17. The culture pH was regulated at 5.0±0.1 with 100% ammonium hydroxide. A 5% (w/v) solution of Struktol J673 antifoam (Qemi International) was added as needed to control foaming. Agitation (maximum 900 rpm) and aeration (ca. 1.0 vvm) were set to maintain dissolved oxygen>20%. Once the initial Cerelose® had been exhausted, a 50% Cerelose® feed containing 2.1 mg·L⁻¹ d-biotin and 2.7×PTM1 trace element solution was initiated to deliver 3 g·L⁻¹·h⁻¹ Cerelose®. The feed rate was increased over the next 42 h to a maximum of 7 g·L⁻¹·h¹ Cerelose®. Aliquots of 1-2 L were removed daily from the vessel to maintain a feasible working volume. The fermentation culture was sampled every 24 h to monitor rPI2_{JO} production.

Analytical methods. SDS-PAGE was done by the method of Laemmli and gels were stained for proteins with GelCode Blue (Pierce). For Western analysis, SDS-PAGE gels were electroblotted onto nitrocellulose membranes (BioRad) using Towbin buffer. Polyclonal rabbit anti-tomato-PI2 primary antibody was hybridized to the blot at 4° C. for 16 h in Tris-buffered saline (TBS) (Sambrook, et al. 1989) with 5% (w/v) dry milk. After rinsing the blot with TBS, alkaline phosphatase-conjugated goat anti-rabbit secondary antibodies (BioRad) were used to hybridize to the blot at room temperature for 1 h in TBS with 5% (w/v) dry milk. Finally, positive hybridization was visualized by developing the blot with 10 mL of 100 mM Tris-HCl buffer (pH 9.5), 100 mM NaCl, 5 mM MgCl₂, 0.33 mg·mL⁻¹ nitro blue tetrazolium, and 0.165 mg·mL⁻¹ 5 bromo-4-chloro-3-indolylphosphate. Trypsin inhibition activity and HPLC quantification of rPI2_{JO} were measured.

B. Results and Discussion

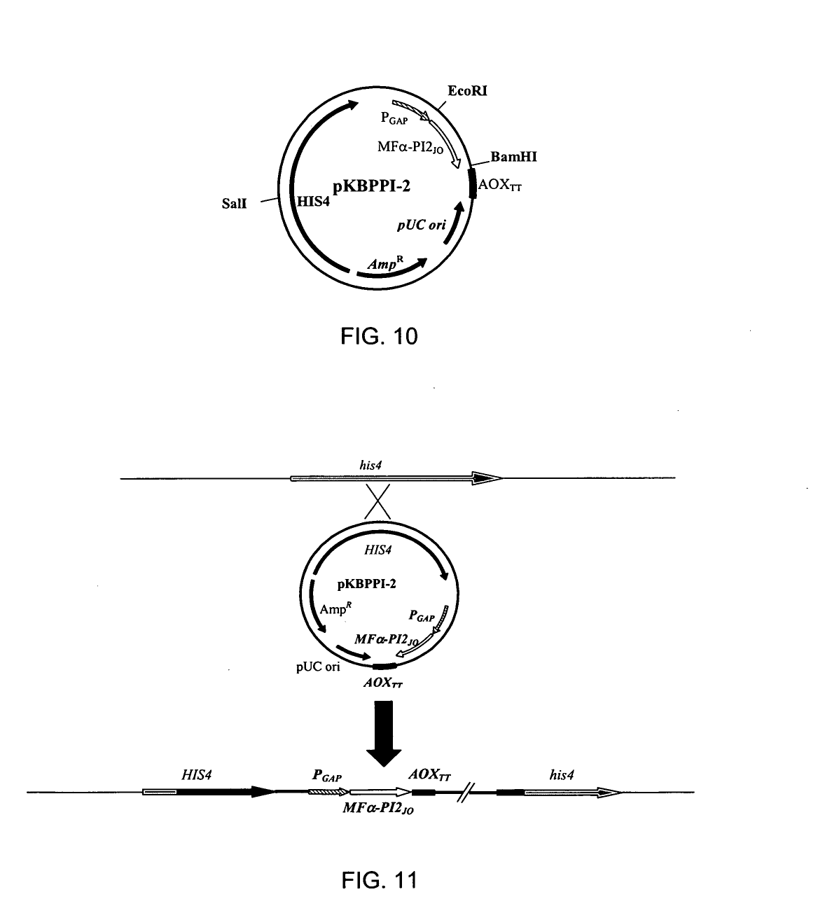

Cloning MFα-PI2_{JO} gene into pIB2 and transformation of P. pastoris. The aim of the present study was to examine whether the constitutive PGAP promoter can direct high-level expression and secretion of rPI2_{JO}. We previously reported that the MFα-PI2_{JO} fusion gene was expressed by P. pastoris GS115 when regulated by the inducible PAOX1 promoter (results not presented). The previously constructed MFα-PI2_{JO} fusion gene was PCR-amplified and sub-cloned into plasmid pIB2 (Sears, et al.) such that its expression would be under the control of the constitutive PGAP promoter, forming plasmid pKBPPI-2 (FIG. 10).

Plasmid pKBPPI-2 was linearized with SalI at the HIS4 locus prior to electroporation into His⁻ GS115 cells. Because pKBPPI-2 lacks a yeast origin of replication that would allow autonomous replication in P. pastoris, transformants that are His⁺ would represent the integration of at least one copy of the linearized plasmid into the his4 locus of the P. pastoris genome (FIG. 11). The presence of the MFα-PI2_{JO} fusion gene in these His⁺ transformants was confirmed by colony PCR using primers a-Factor seq and 3, AOX1 seq (data not shown).

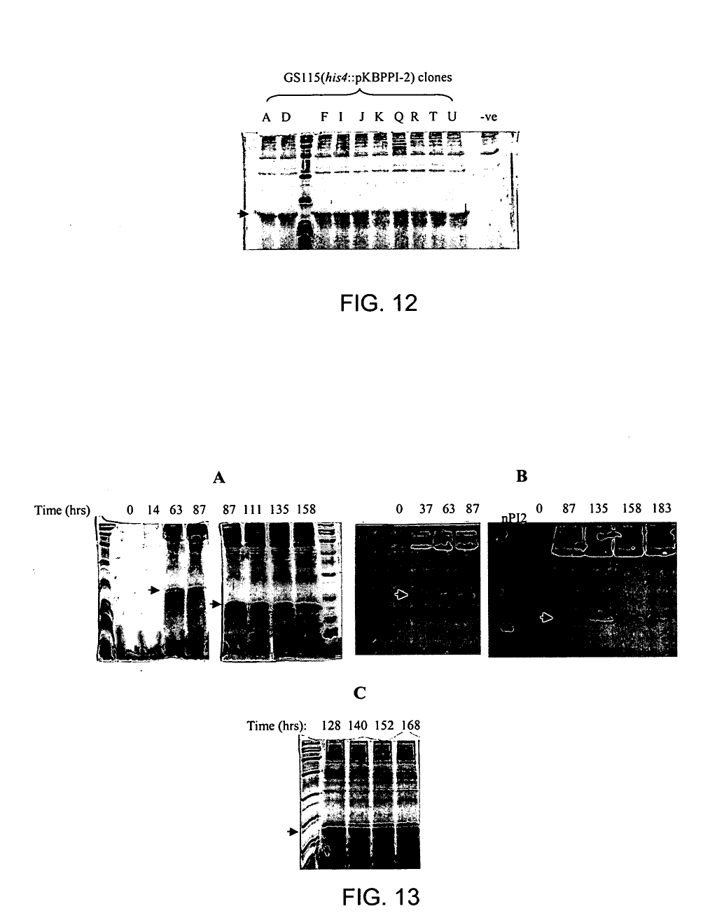

Screening for His⁺ P. pastoris transformants that secreted rPI2_{JO} constitutively. Ten His⁺ GS115(his4::pKBPPI-2) transformants were grown in 5 mL YPD media in culture tubes incubated at 30° C. with shaking at 270 rpm for 3 days. The culture supernatants were analyzed for the accumulation of rPI2_{JO}. An SDS-PAGE analysis showed that all of these His⁺ GS115(his4::pKBPPI-2) transformants secreted a protein with apparent MW similar to that of PI2_{JO}; such a protein was not secreted by the negative control GS115(his4::pIB2) clone (FIG. 12). The secretion of rPI2_{JO} was detectable on day 1 and reached a maximum level after day 2. The rPI2_{JO} production levels by these 10 clones were very similar. Even when grown in 250-mL culture flasks with 50 mL of YPD media, the rPI2_{JO} production levels by these clones were not significantly different from those in the culture tube experiment. Thus, one of the GS115(his4::pKBPPI-2) transformants, clone U, was randomly chosen for rPI2_{JO} production in fermentative studies.

Constitutive expression of rPI2_{JO} by fermentation. The fermentation process was run initially in batch mode, followed by fed-batch mode, with Cerelose® (CPC International) as the major carbon source. The fermentation was not supplemented with pure oxygen. Agitation (maximum 900 rpm) and aeration (ca. 1.0 vvm) were set to maintain the dissolved oxygen level above 20%. Despite maximum agitation and aeration, the dissolved oxygen in fermentation #PPI2 fell below 20% after about 110 hours post inoculation. The maximum feed rate for Cerelose® (7 g·L⁻¹·h⁻¹) was not lowered in an effort to raise the DO in this fermentation. Shortly after the DO fell to near 0%, the cell density reached a plateau. In fermentation #PP17, the feed rate was adjusted to maintain a DO≧20% once aeration and agitation had reached their maximum set points. The maximum feed rate in #PP17 was 7 g·L⁻¹·h⁻¹ falling to as low as 3.6 g·L⁻¹·h⁻¹ once the feed rate was used to maintain the DO at 20%.

The culture supernatant was analyzed for rPI2_{JO} by SDS-PAGE and Western blot. Western blots confirmed the accumulation of rPI2_{JO} in the culture supernatant, and rPI2_{JO} was detectable as early as 37 hours post-inoculation (FIGS. 13A & B). SDS-PAGE analysis showed that the concentration of rPI2_{JO} in the culture supernatant increased concurrently with the increase in biomass. However, rPI2_{JO} was not the major protein in the culture supernatant of this constitutive expression system, while, rPI2_{JO} was the major protein produced by the methanol-inducible P_{AOX1}-based expression system (FIG. 13C). In order to quantify the rPI2_{JO} productivity by the P_{GAP}-based expression system, the two constitutive fermentations of clone U (#PP12 and #PP17) were processed. Biomasses were removed by centrifugation followed by filtration through a 0.2-μm PES membrane, and the rPI2_{JO} in the cell-free fermentation broth was purified by cation-exchange chromatography. About 2.1 g of pure rPI2_{JO} was produced per fermentation (Table 5).

TABLE 5
rPI2_{JO} productivity by the constitutive P. pastoris GS115(his4::pKBPPI-2)
clone U cultured by fermentation

| | Total vol. of | | Total mass of | |
|---|---|---|---|---|
| Fermentation | 0.2-μm | [rPI2_{JO}] | rPI2_{JO} | Length of |
| No. | permeate (L) | (mg/L) | produced (mg) | fermentation (h) |
| #PP12 | 14.3 | 150 | 2145 | 211 |
| #PP17 | 10.1 | 217 | 2191 | 214 |

In summary, we have shown that rPI2_{JO} can be expressed constitutively in P. pastoris by fed-batch fermentation. The rPI2_{JO} concentration in the cell-free fermentation culture supernatant (ca. 150-200 mg·L⁻¹) The constitutive P_{GAP} expression system can be operated as continuous fermentation (Schilling, B. M., Goodrick, J. C., and Wan, N.C. (2001) Biotechnol. Prog. 17, 629-633; Vassileva, A., Chugh, D. A., Swaninathan, S., and Khanna, N. (2001) J. Biotechnol. 88, 21-35) and therefore may save a considerable amount of money, time and effort in setting up large-scale fermentations run in batch or fed-batch mode. We have recently tested a semi-continuous fermentation of P. pastoris GS115(his4::pKBPPI-2) clone U. rPI2_{JO} concentration in the fermentation broth was successfully maintained at 210-250 mg·L⁻¹ for 17 days (data not shown).

III. The Use of High Gene Dosage to Increase the Level of Expression

In this experiment we described the use of a combination of genetic/molecular biology strategies to improve constitutive expression of rPI2. These strategies included optimizing the 5′ untranslated region of the PI2 transcript, integrating the PI2 expression cassette at a different locus of P. pastoris genome, and increasing the expression cassette copy number. Our results suggest that the gene copy number plays a major role in improving rPI2 expression level. A new recombinant strain, KS4X2, transformed with 4 copies of the PI2 gene regulated by the constitutive P_{GAP} promoter was constructed. An expression level of ca. 450 mg·L⁻¹ rPI2 was achieved in fed-batch fermentation.

A. Experimental Procedures

Materials. All reagents were of the highest purity available and were purchased from Sigma, Aldrich, and Fisher Scientific unless otherwise noted. PCR primers were purchased from Integrated DNA Technologies. Taq DNA polymerase, restriction endonucleases, and T4 DNA ligases were purchased from Invitrogen, New England Biolabs, and Roche, respectively.

Host strains and media. Escherichia coli strains DH5α and TOPO10 (Invitrogen) grown in LB medium (Sambrook, et al., 1989) were used for propagation of recombinant plasmids. Ampicillin and Zeocin were used at 50 μg·mL⁻¹ and 25 μg·mL⁻¹, respectively, when required. Pichia pastoris strain GS115 (His⁻, where His⁻ represents an auxotrophic phenotype that requires histidine supplementation) and KM71H (His⁺, a histidine prototroph) (Invitrogen) were used for expressing rPI2_{JO} (GenBank accession no. L37519) P. pastoris was normally cultured in YPD medium (1% (w/v) yeast extract, 2% (w/v) peptone, and 2% (w/v) dextrose). YPD plates with 100 μg·mL⁻¹ Zeocin and 1 M of sorbitol were used for selection of P. pastoris cells transformed with the PI2 expression plasmids.

Construction of the P_{GAP}-MFα-PI2_{JO}-AOX_{TT} expression cassette by crossover PCR. The P_{GAP}-MFα-PI2_{JO}-AOX_{TT} expression cassette with a modified 5′ UTR sequence was constructed by the crossover PCR technique of Link et al. (Link, A. J., Phillips, D., and Church, G. M. (1997) J. Bacteriol. 179, 6228-6237). In the first step, two different 25-μL asymmetric PCRs were used to generate the PGAP fragment and the MFα-PI2_{JO}-AOX_{TT} fragment. Plasmid pKBPPI-2 was used as template for amplifying the P_{GAP} fragment, and plasmid pKBPPI-1 was used as template for amplifying the MFα-PI2-AOX_{TT} fragment. The asymmetric PCR reactions contained 20 mM Tris-HCl (pH 8.4), 50 mM KCl, 1.5 mM MgCl₂, 200 μM dNTPs, 2.5 U of Taq DNA polymerase, and the primer pairs (Table 6) were in a 10:1 molar ratio (600 nM Pgap-No plus 60 nM Pgap-Ni or 600 nM MFα-PI2-Co plus 60 nM MFα-PI2-Ci). The 30-cycle PCR thermal profile was 30 s at 94° C., 30 s at 58° C. and 45 s at 72° C., followed by a 10 min soak at 72° C. and a hold at 4° C. In the second step, 1 μL of each of the asymmetric PCR mixtures and 600 nM each of the two outside primers were mixed together. The P_{GAP} fragment and the MFα-PI2-AOX_{TT} fragment would anneal at their complementary region and be amplified by PCR with the following thermal profile: (i) 5 cycles of 30 s at 95° C., 30 s at 58° C. and 60 s at 72° C., (ii) 30 cycles of 30 s at 95° C., 30 s at 61° C. and 60 s at 72° C. (iii) a 10 min soak at 72° C. and a hold at 4° C. The 1.6-kb fusion product was gel purified, and its nucleotide sequence was confirmed by DNA sequencing using primers a-Factor seq, Sequen1, Sequen2, and Sequen3 (Table 6).

TABLE 6
Oligonucleotide primers used in this study

| | |
|---|---|
| Primer | Sequence* |

| | |
|---|---|
| Pgap-No | 5′-GTCAGCCAGATCTTTTTTGTAGAAATG-3′ |
| Pgap-Ni | 5′-AATCTCATCGTTTCGAAATAGTTGTTCAATTGATTGAAATAGG-3′ |
| MFα-PI2-Ci | 5′-ACAACTATTTCGAAACGAGATTTCCTTCAATTTTTACTGC-3′ |
| MFα-PI2-Co | 5′-AGGAGTAGAAACATTTTGAAGCTATGG-3′ |
| a-Factor seq | 5′-TACTATTGCCAGCATTGCTGC-3′ |
| Sequen1 | 5′-CTAAAGTGCAAGCCTTCG-3′ |
| Sequen2 | 5′-GGATCTGAATAGCGCCGT-3′ |
| Sequen3 | 5′-CTATTATTGCCAGCGACG-3′ |
| Pgap-SDM-F | 5′-CGCCCGTTACCGTCCCTAGAAATTTTACTCTG-3′ |
| Pgap-SDM-R | 5′-CAGAGTAAAATTTCTAGGGACGGTAACGGGCG-3′ |
| Pgap-UP | 5′-AGCAGCAGATTACGCGCAG-3′ |

*Primer Pgap-No has a BglII site (underlined). The bold face indicates the complementary region of primers Pgap-Ni and MFα-PI2-Ci. The ATG start codon of MFα-PI2 in primer MFα-PI2-Ci was highlighted.

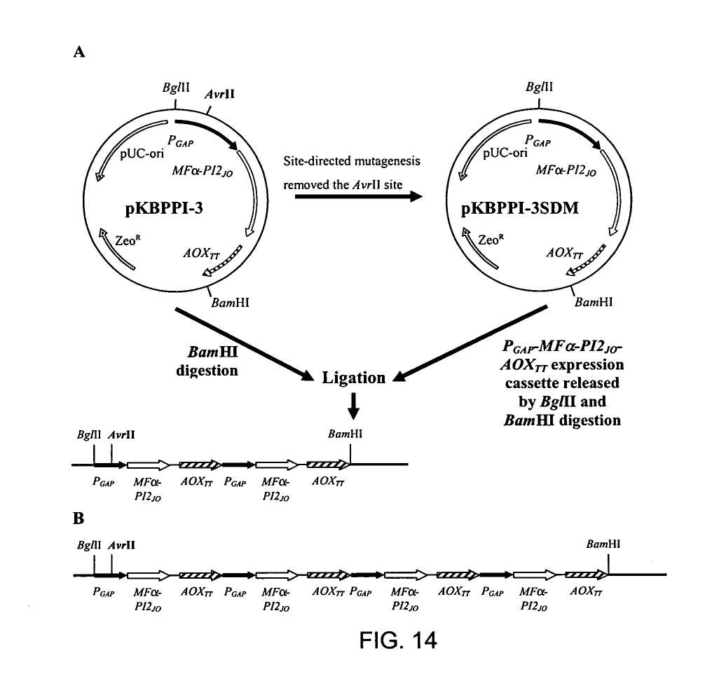

Construction ofplasmids pKBPPI-3 andpKBPPI-3SDM. The P_{GAP}MFα-PI2_{JO}-AOX_{TT} expression cassette constructed by crossover PCR was digested with BglII and BamHI, followed by ligation with plasmid pPICZαA (Invitrogen) that was previously digested by BglII and BamHI. The resulting plasmid was pKBPPI-3 (FIG. 14). The AvrII site within P_{GAP} promoter of pKBPPI-3 was removed by using the Quikchange site-directed mutagenesis kit (Stratagene) according to the manufacturer's instruction. Primers Pgap-SDM-F and Pgap-SDM-R (Table 6) were used to introduce a single base deletion in pKBPPI-3, resulting in pKBPPI-3SDM (FIG. 14). Removal of the AvrII site in pKBPPI-3SDM was confirmed by DNA sequencing with primers Pgap-UP (Table 6).

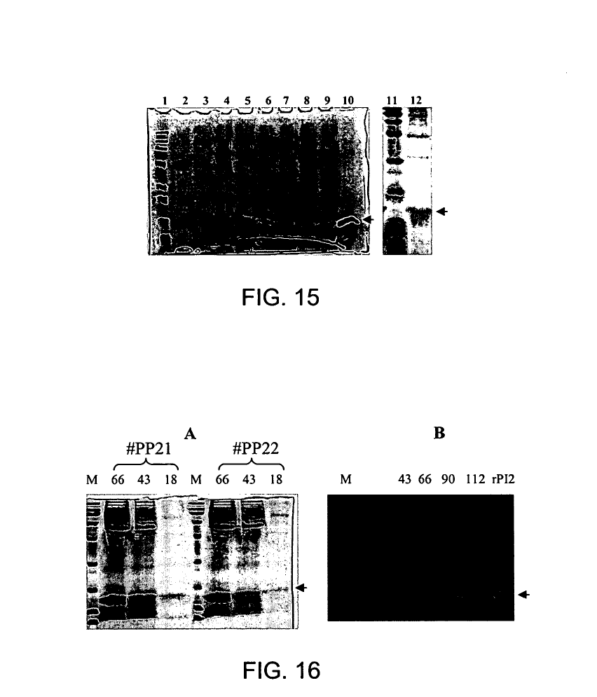

Construction of plasmids that carried multiple tandem copies of the expression cassettes. Restriction digestion of pKBPPI-3SDM with BglII and BamHI resulted in the release of the entire rPI2_{JO} expression cassette that contained the site-direct mutated P_{GAP} promoter (FIG. 15), which was subsequently gel-purified. To achieve two tandem copies of the rPI2_{JO} expression cassette in a single vector, pKBPPI-3 was linearized with BamHI, and ligated with the gel-purified rPI2_{JO} expression cassette cut out from pKBPPI-3SDM. The resulting plasmid, pKBPPI-4, contained a unique BglII site at the 5′ end of the PGAP promoter of the first cassette and a single BamHI site at the 3′ end of the AOX_{TT} of the second cassette (FIG. 15). Also, the AvrII site was present only in the PGAP promoter of the first expression cassette. A procedure similar to that described for the construction of pKBPPI-4 was used to construct plasmids pKBPPI-5 and pKBPPI-6, which contained 4 and 7 tandem copies of the rPI2_{JO} expression cassettes, respectively. SEQ ID NO4 provides the sequence of the MFcI-PI2 fusion gene sequence in pKBPPI-3, pKBPPI-4, pKBPPI-5, and pKBPPI-6; MFα secretion signal is underlined and PI2_{JO} is bold.

P. pastoris transformation and culture tube expression experiment. Cells of P. pastoris KM71H and GS115 were transformed with 2.5 to 5 μg of AvrII-linearized pKBPPI-3, pKBPPI-4, pKBPPI-5, and pKBPPI-6 by electroporation as outlined in the manual of Invitrogen's EasySelect™ Pichia Expression Kit. Transformants were selected on YPD agar plates containing 1 M sorbitol and 100 μg·mL⁻¹ of Zeocin. After three days of incubation at 30° C., possible transformants were streaked for purity on YPD plus Zeocin agar plates. Zeocin-resistant transformants were then screened for rPI2_{JO} production in liquid cultures. Twenty μL of selected transformants that had grown overnight in YPD medium at 30° C. were used to inoculate 3 mL fresh YPD media. The cultures were incubated at 30° C. for 3 days with vigorous shaking at 270 rpm. Every 24 h, 100 μL of culture was sampled to analyze for rPI2_{JO} production by SDS-PAGE analysis.

Constitutive expression of rPI2_{JO} by fed-batch fermentation. Transformants KS4X2 and KS7X1 were chosen to test for rPI2_{JO} production by fed-batch fermentation. Single colonies of each transformant were used to inoculate 300 mL YPD with 75 μg·mL⁻¹ Zeocin and were incubated for 23 h at 30° C., 200 rpm. Each seed culture was used to inoculate a 14-L BioFlo 3000 vessel (New Brunswick Scientific Co.) equipped with two Rushton impellers and four baffles, containing 8 L Basal Salt Medium, 40 g·L⁻¹ Cerelose®, 0.8 mg·L⁻¹ d-biotin, and 1×PTM1 trace element solution (Stratton, J., Chiruvolu, V., and Meagher, M. (1998) in Pichia protocols (Higgins, D. R., and Cregg, J. M., eds) Vol. 103, pp. 107-120, Humana Press Inc., Totowa). The production culture temperature was maintained at 30° C. for the first 42 h of growth, and then at 23° C. for the remainder of the fermentation. The culture pH was regulated at 5.0±0.1 with 100% ammonium hydroxide. A 5% (w/v) solution of Struktol J673 antifoam (Qemi International) was added as needed to control foaming. Agitation (maximum 900 rpm) and aeration (ca. 1.0 vvm) were set to maintain dissolved oxygen above 20%. Once the initial Cerelose® had been exhausted, a 50% (w/v) Cerelose® feed containing 2.1 mg·L⁻¹ d-biotin and 2.7×PTM1 trace element solution was initiated to deliver 3 g·L⁻¹·h⁻¹ Cerelose®. The feed rate was increased over the next 19 h to a maximum of 7 g·L⁻¹·h⁻¹ Cerelose®. Aliquots of 1 to 2 L were removed daily from the vessel to maintain a feasible working volume. The fermentation culture was sampled every 24 h to monitor rPI2_{JO} production.

Resin purification of fermentation samples for HPLC analysis. When the maximum feed rate had been achieved in fed-batch fermentations, and approximately every 24 h thereafter, 200-300 mL culture broth was removed from the fermentor and centrifuged at 10,000×g for 15 min. The supernatant was diluted with deionized H₂O to a conductivity of less than 8 mS, and the pH of the diluted material was adjusted to 4.0±0.3 with 1 N citric acid. This material was column-purified and was stored at 4-8° C. until analysis by HPLC. Analytical methods. SDS-PAGE was done by the method of Laemmli and gels were stained for proteins with GelCode Blue (Pierce). For Western analysis, SDS-PAGE gels were electroblotted onto a nitrocellulose membrane (BioRad) using Towbin buffer (Towbin, et al.). Polyclonal rabbit anti-potato-PI2 primary antibody (Maine Biotechnology) was hybridized to the blot for 16 h in Tris-buffered saline (TBS) (Sambrook, et al.) with 5% (w/v) dry milk. After rinsing the blot with TBS, alkaline phosphatase-conjugated goat anti-rabbit secondary antibodies (BioRad) were used to hybridize to the blot at room temperature for 1 h in TBS with 5% (w/v) dry milk. Finally, positive hybridization was visualized by developing the blot with 10 mL of 100 mM Tris-HCl buffer (pH 9.5), 100 mM NaCl, 5 mM MgCl₂, 0.33 mg·mL⁻¹ nitro blue tetrazolium, and 0.165 mg·mL⁻¹ 5 bromo-4-chloro-3-indolylphosphate. HPLC quantification of rPI2_{JO} was performed.

B. Results and Discussion

Sequence analysis of the P_{GAP}-MFα-PI2_{JO}-AOX_{TT} expression cassette. We previously described that rPI2_{JO} was expressed and secreted by P. pastoris when the fusion gene MFα-PI2_{JO} was cloned into plasmid pIB2 (Sears, et al.), creating plasmid pKBPPI-2. MFα-PI2_{JO} was regulated by the constitutive PGAP promoter. An expression level of 217 mg·L⁻¹ was achieved by fed-batch fermentation. pIB2 has a polylinker sequence different from other P_{GAP}-containing plasmids. In a P. pastoris review article by Sreekrishna (Sreekrishna, K. (1993) in Industrial microorganisms: basic and applied molecular genetics. (Baltz, R. H., Hegeman, G. D., and Skatrud, P. L., eds), pp. 119-126, American Society of Microbiology, Washington D.C.), it was suggested that an optimal 5′ UTR of the transcript may be critical for protein expression-in P. pastoris (Sreekrishna, 1993). Sreekrishna also suggested that for optimal expression, the composition of the nucleotides in the −1 to −25 positions relative to the AUG start codon should be greater than 63% A+U. When the nucleotide sequence of pKBPPI-2 was examined and compared to Invitrogen's P. pastoris expression plasmids pPICZαA (contains P_{AOX1}) and pGAPZαA (contains P_{GAP}), we noticed that the 5′ UTR sequence of plasmid pGAPZαA was more similar to that of pPICZαA than to pKBPP-2 in the region directly 5′ to the AUG start codon (Table 7, in bold face and underlined). Also, both of the Invitrogen's plasmids have higher A+U content than pKBPPI-2 in the −1 to −25 positions relative to the start codon. Expression of rPI2_{JO} regulated by the P_{GAP} promoter may be increased if we modify the 5′ UTR to a nucleotide sequence more similar to that of P_{AOX1}.

Construction of a new P_{GAP}-MFα-PI2_{JO}-AOX_{TT} expression cassette with modified 5′ UTR. We optimized the 5′ UTR of the PI2 expression cassette by crossover PCR as described in the Experimental Procedures section. The final crossover PCR product is a new PGApMFa-PI2_{JO}-AOX_{TT} expression cassette that has a 5′ UTR with higher A+U content in the first 25 nucleotides directly 5′ to ATG, and is identical to that of pGAPZαA (Table 7). Several nucleotides that were originally present in this region in plasmid pKBPPI-2 were deleted (Table 7, labeled by asterisks). Also, this new expression cassette was flanked by BglII and BamHI restriction sites that are not present on pKBPPI-2. After digesting this new expression cassette by BglII and BamHI, it was ligated into the BglII and BamHI sites of plasmid pPICZαA (Invitrogen), resulting in the expression plasmid pKBPPI-3 that has a Zeocin-resistant (ZeoR) selection marker (FIG. 14).

TABLE 7

Comparison of the partial nucleotide sequence of plasmids that have
been used or can be used to express PI2.

| | |
|---|---|
| | A + T content of the |
| MFα start | firts 25 nuclotides 5′ |
| condon | to ATG |

P _{GAP} promoter 5′ UTR

| | | | |
|---|---|---|---|
| | ****** * | | |
| pKBPPI-2: | AATCAATTGAACAACTATCAAGAATTCCGAAACG | -ATG••••• | 64% |
| pGAPZαA: | AATCAATTGAACAACTAT------TTC-GAAACG | -ATG••••• | 68% |
| pKBPPI-3: | AATCAATTGAACAACTAT------TTC-GAAACG | -ATG••••• | 68% |

P _{AOXI} promoter 5′ UTR

| | | | |
|---|---|---|---|
| PICZαA: | TCAAAAAACAACTAATTA------TTC-GAAACG | ATG••••• | 76% |

Cloning multiple copies of P_{GAP}-MFα-PI2_{JO}-AOX_{TT} and transformation of P. pastoris.

Increasing the copy number of the heterologous gene is one of the methods that have been used successfully to improve the production of recombinant proteins in P. pastoris (Vassileva, et al.; Sreekrishna; Paus, et al.; Clare). Therefore, we constructed a series of expression plasmids that contained tandem copies of the P_{GAP}-MFα-PI2_{JO}-AOX_{TT} expression cassette with modified 5′ UTR as described in the Experimental Procedures section (FIG. 15). The resulting plasmids pKBPPI-4, -5, and -6 contained 2, 4, and 7 copies of the new expression cassettes, respectively, and allowed us to examine the effect of gene dosage on rPI2_{JO} expression.

Previously, the constitutive expression plasmid pKBPPI-2 was integrated into the his4 locus instead of the P_{GAP} locus. Both PGAP and his4 loci of P. pastoris have been used successfully for protein expression but Sreekrishna noticed occasional loss of the lacZ expression cassette integrated at the his4 locus due to recombination between the chromosomal his4 and the dominant HIS4 marker on the expression cassette while retaining the His⁺ phenotype. We decided to investigate the effect of integration site in rPI2_{JO} expression by integrating our new expression plasmids at the P_{GAP} locus in this study. Plasmids pKBPPI-3, -4, -5, and -6 were individually transformed into P. pastoris GS115 and KM71H. Prior to transformation, the plasmids were linearized by AvrII at PGAP in order to increase the integration frequency (Orr-Weaver, T. L., Szostak, J. W., and Rothstein, R. J. (1981) Proc. Natl. Acad. Sci. USA. 78, 6354-6358) into the P_{GAP} locus of P. pastoris genome. Because of this reason, the AvrII sites in all but the first expression cassettes in these plasmids were mutated by a single base deletion (FIG. 15). All of these expression plasmids lack a yeast origin of replication that would allow autonomous replication in P. pastoris. Therefore, Zeocin-resistant transformants would result only from the integration of at least one copy of the linearized plasmid into the P_{GAP} locus. These Zeo® transformants were directly screened for rPI2_{JO} production.

Screen ing for Zeo® P. pastoris transformants. Zeo® transformants of each expression plasmid were grown in culture tubes containing YPD media. The cultures were incubated at 30° C. with shaking at 270 rpm for 3 days, and the culture supernatants were analyzed for the accumulation of rPI2_{JO}. An SDS-PAGE analysis showed that most of these Zeo® transformants secreted rPI2_{JO} (FIG. 15). rPI2_{JO} was detectable on day 1 and reduced slightly in day 2 and 3. The production level of rPI2_{JO} increased progressively with the copy number of the expression cassettes (FIG. 15). This effect is most likely due to the increase in number of expression cassettes, rather than due to the plasmid integration at the P_{GAP} locus and the optimization of the 5′ UTR. Two clones originated from pKBPPI-3 transformation in this study (FIG. 15, lanes 2 and 3) appeared to secrete less rPI2_{JO} than the previous P_{GAP} recombinant strain that contained 1 copy of the expression cassette without an optimized 5′ UTR and integrated at the his4 locus (FIG. 15, lane 12). There is no apparent difference in rPI2_{JO} production between transformants originated from P. pastoris GS115 or KM71H. Since strain KM71H is a histidine prototroph and its transformants do not require histidine supplementation during fermentation, two KM71H transformants were selected for fed-batch fermentations. The first clone selected was KS4X2, a pKBPPI-5 transformant that contains 4 copies of the expression cassette, while the second clone selected was KS7X1 which originated from the pKBPPI-6 transformation and contains 7 copies of the expression cassette.

Constitutive expression of rPI2_{JO} by fermentation. The fermentations of clones KS4X2 (fermentation #PP21) and KS7X1 (fermentation #PP22) were run initially in batch mode, followed by fed-batch mode, with Cerelose® as the major carbon source. The fermentations were not supplemented with pure oxygen. Agitation (maximum 900 rpm) and aeration (ca. 1.0 vvm) were set to maintain the dissolved oxygen level above 20%. The feed rate was adjusted to maintain a DO≧20% once aeration and agitation had reached their maximum set points. SDS-PAGE and Western analyses showed that rPI2_{JO} was accumulated in the culture supernatants of the 2 fermentations, as in the culture tube experiments (FIG. 16).

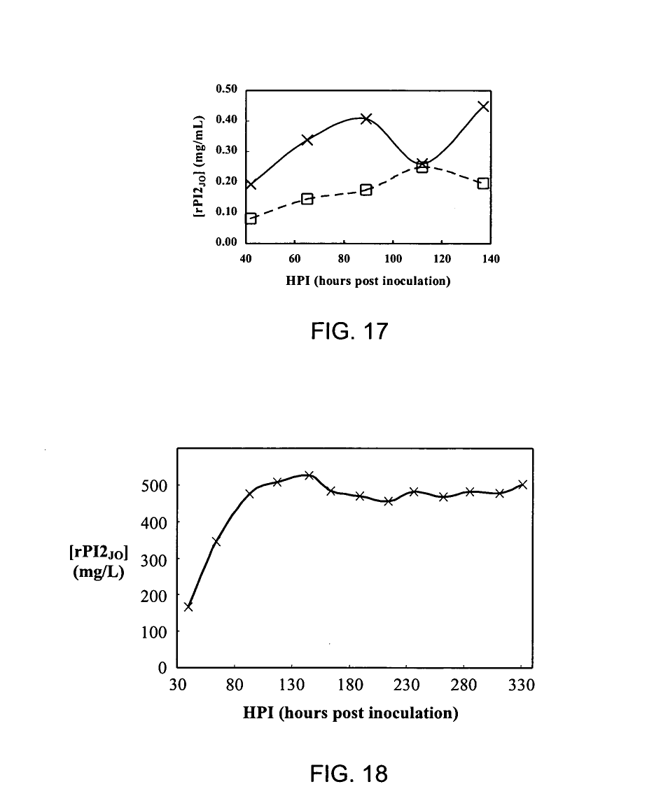

Clone KS7X1 secreted less rPI2_{JO} than clone KS4X2 over the entire time course of the fermentation, as measured by HPLC of column-purified samples (FIG. 17). The decrease in protein yield in clone KS7X1 is not surprising since it has been observed that an excess copy number of expression cassette reduces secretory protein yields (Brierley, R. A. (1998) in Pichia protocols (Higgins, D. R., and Cregg, J. M., eds) Vol. 103, pp. 149-177, Humana Press, Inc., Totowa, N.J.; Thill, G. P., Davis, G. R., Stillman, C., Holtz, G., Brierley, R., Engel, M., Buckholtz, R., Kinney, J., Provow, S., Vedvick, T., and Siegel, R. S. (1990) in Proceedings of the 6th International Symposium on Genetics of Microorganisms, vol. II (Heslot, H., Davies, J., Florent, J., Bobichon, L., Durand, G., and Penasse, L., eds), pp. 477-490, Société Francaise de Microbiolgie, Pairs; Scorer, C. A., Buckholz, R. G., Clare, J. J., and Romanos, M. A. (1993) Gene 136, 111-119). rPI2_{JO} concentration in fermentation #PP21 decreased at 112 h post-inoculation but increased again at 138 h (FIG. 17). This decrease was also observed on SDS-PAGE gel (data not shown).

In summary, we have improved the constitutively expressed rPI2_{JO} level in P. pastoris grown in fed-batch fermentation from ca. 200 mg·L⁻¹ to a maximum production of ca. 450 mg·L⁻¹ by increasing the gene copy number. From a single fed-batch fermentation of KS4X2 that yielded approximately 10 L filtrate, we were able to recover approximately 4 g rPI2_{JO} during 144 h fermentation time, or 2.78 mg rPI2_{JO}·L⁻¹·h⁻¹. Strain GS115(his4::pKBPPI-2), which has 1 copy of the expression cassette, only produced 1.01 mg rPI2_{JO}·L⁻¹·h⁻¹ from a similarly run fed-batch fermentation (#PP17). In addition, the constitutive P_{GAP} expression system can be operated as continuous fermentation (Vassileva; Goodrick, J. C., Xu, M., Finnegan, R., Schilling, B. M., Schiavi, S., Hoppe, H., and Wan, N.C. (2001) Biotech. Bioeng. 76, 492-797) and therefore may increase overall efficiency of large-scale fermentations. We have demonstrated the feasibility of continuous fermentation with strain KS4X2; rPI2_{JO} expression was successfully maintained at 480 to 500 mg·L⁻¹ for 13 days (FIG. 18).

The increase in rPI2_{JO} expression by clone KS4X2, a recombinant strain that has 4 copies of the expression cassettes, is lower than perhaps expected. We had anticipated a 3 to 4 fold increase in productivity to 600-800 mg·L⁻¹ rPI2_{JO} from clone KS4X2 becausein numerous P. pastoris studies, heterologous protein expression levels were increased linearly in relation to the gene copy number. For example, the yield of secreted aprotonin increased 7 times to 900 mg·L⁻¹ with 5 copies of the gene (Thill, et al.), the yield of secreted IGF-1 increased 5 times to 500 mg·L⁻¹ with 6 copies of the gene (Brierley, R. A., Davis, G. R., and Holtz, G. C. (1994) U.S. Pat. No. 5,324,639; Brierly, 1998), and secreted hepatitis B surface antigen expression increased 4 times when the gene copy number was increased from 1 to 4. Since we observed a decrease in rPI2_{JO} expression in the 7-copy clone KS7X1, it is apparent that the linear relationship of gene copy number to expression level does not hold with the expression of PI2. Additionally, since the recombinant strains originating from pKBPPI-3 transformation secreted less rPI2_{JO} than our previous strain GS115(his4::pKBPPI-2) that contained 1 copy of the expression cassette integrated at the his4 locus (FIG. 4), apparently gene integration site can play a role in rPI2 expression and the nature of the targeted integration site apparently does not predict the expression level. Indeed, some reports did not observe the direct additive relationship between gene dosage and expression level. For instance, mouse epidermal growth factor expression increased only 13 fold to 450 mg·L⁻¹ with 19 copies of the gene (Clare et al., 1991).

In conclusion, we have demonstrated the feasibility to improve constitutive expression level of rPI2 in P. pastoris by increasing the gene copy number. A P. pastoris recombinant strain, KS4X2, was constructed by this strategy, and it expressed and secreted rPI2 in excess of the previously reported recombinant strain GS115(his4::pKBPPI-2).

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

We claim:

1. A method of producing potato proteinase inhibitor II, comprising the steps of:
(a) culturing a microbial host, wherein said microbial host comprises an expression unit comprising a DNA segment encoding potato proteinase inhibitor II heterologous to the microbial host, under conditions in which said potato proteinase inhibitor II is expressed into the culture medium; and
(b) recovering not less than 500 mg of said potato proteinase inhibitor II per liter of culture medium.

2. A method as described in claim 1, wherein said microbial host is selected from the group consisting of bacteria and fungi.

3. A method as described in claim 1, wherein said bacteria host is Escherichia coli.

4. A method as described in claim 1, wherein said fungal host is Pichia pastoris.


SEQUENCE LISTING (4 sequences)

SEQ ID NO: 1 - DNA, 378 bp, Escherichia coli
atggcgaaggcttgcactttagaatgtggtaatcttgggtttgggatatgcccacgttcagaaggaagtccggaaaatcgcatatgcaccaactgttgtgcaggttataaaggttgcaattattatagtgcaaatggggctttcatttgtgaaggacaatctgacccaaaaaaaccaaaagcatgccccctaaattgcgatccacatattgcctactcaaagtgtccccgttcagaaggaaaatcgctaatttatcccaccggatgtaccacatgctgcacagggtacaagggttgctactatttcggtaaaaatggcaagtttgtatgtgaaggagagagtgatgagcccaaggcaaatatgtaccctgcaatgtag

SEQ ID NO: 2 - DNA, 378 bp, Escherichia coli
atggcgaaggcttgcactttagaatgtggtaatcttgggtttgggatatgcccacgttcagaaggaagtccggaaaatcgcatatgcaccaactgttgtgcaggttataaaggttgcaattattatagtgcaaatggggctttcatttgtgaaggagagtctgacccaaaaaaaccaaaagcatgcccccggaattgcgatccacatattgcctactcaaagtgtccccgttcagaaggaaaatcgctaatttatcccaccggatgtaccacatgctgcacagggtacaagggttgctactatttcggtaaaaatggcaagtttgtatgtgaaggagagagtgatgagcccaaggcaaatatgtaccctgcaatgtag

SEQ ID NO: 3 - DNA, 855 bp, Escherichia coli
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatggcgaaggcttgcactttagaatgtggtaatcttgggtttgggatatgcccacgttcagaaggaagtccggaaaatcgcatatgcaccaactgttgtgcaggttataaaggttgcaattattatagtgcaaatggggctttcatttgtgaaggacaatctgacccaaaaaaaccaaaagcatgccccctaaattgcgatccacatattgcctactcaaagtgtccccgttcagaaggaaaatcgctaatttatcccaccggatgtaccacatgctgcacagggtacaagggttgctactatttcggtaaaaatggcaagtttgtatgtgaaggagagagtgatgagcccaaggcaaatatgtaccctgcaatgtag

SEQ ID NO: 4 - DNA, 630 bp, Pichia pastoris
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagcgaaggcttgcactttagaatgtggtaatcttgggtttgggatatgcccacgttcagaaggaagtccggaaaatcgcatatgcaccaactgttgtgcaggttataaaggttgcaattattatagtgcaaatggggctttcatttgtgaaggagagtctgacccaaaaaaaccaaaagcatgcccccggaattgcgatccacatattgcctactcaaagtgtccccgttcagaaggaaaatcgctaatttatcccaccggatgtaccacatgctgcacagggtacaagggttgctactatttcggtaaaaatggcaagtttgtatgtgaaggagagagtgatgagcccaaggcaaatatgtaccctgcaatgtag